(19) United States
(12) Patent Application Publication
(10) Pub. No.: US 2024/0216356 A1
(43) Pub. Date: Jul. 4, 2024

(54) TASTE MASKED COMPOSTIONS OF 2,4,6-TRIFLUORO-N-[6-(1-METHYL-PIPERIDINE-4-CARBONYL)-PYRIDIN-2-YL]-BENZAMIDE HEMISUCCINATE, AND ORALLY DISENTEGRATING TABLET COMPRISING THE SAME

(71) Applicant: Eli Lilly and Company, Indianapolis, IN (US)

(72) Inventors: Matthew Carl Allgeier, Fishers, IN (US); Adam Shane Butterbaugh, Greenwood, IN (US); Robert Louis Ternik, Fishers, IN (US)

(21) Appl. No.: 18/557,666

(22) PCT Filed: May 6, 2022
(86) PCT No.: PCT/US2022/028003

§ 371 Date: Oct. 27, 2023

Related U.S. Application Data
(60) Provisional application No. 63/185,554, filed on May 7, 2021.

(51) Int. Cl. A61K 31/4545; A61K 9/00; A61K 9/20; A61K 9/28; A61K 9/50

(52) U.S. Cl. CPC A61K 31/4545; A61K 9/0056; A61K 9/2081; A61K 9/2095; A61K 9/2813; A61K 9/282; A61K 9/2846; A61K 9/5015; A61K 9/5047

(57) ABSTRACT

The present disclosure provides a novel palatable pharmaceutical composition in the form of taste-masked 2,4,6-trifluoro-N-[6-(1-methylpiperidine-4-carbonyl)-2-pyridyl]benzamide hemisuccinate, and orally disintegrating tablets comprising the same. The taste-masked orally disintegrating tablets of this invention will significantly reduce the potently bitter taste of lasmiditan, and enable administration of this product form to migraine patients, in particular pediatric patients and those suffering from nausea due to migraine attacks.

The embodiments of the present inventions relate to the fields of pharmaceutical composition chemistry and provide coated compositions, processes and formulations for orally disintegrating preparations of 2,4,6-trifluoro-N-[6-(1-methyl-piperidine-4-carbonyl)-pyridin-2-yl]-benzamide hemi-succinate salt, a 5-HT_{IF} receptor agonist, and product forms made by these processes, and uses thereof for rapid oral administration of lasmiditan for the treatment of migraine.

In October 2019, the US FDA approved the use of REYVOW® (lasmiditan) 50 and 100 mg tablets for the acute on-demand treatment of migraine with or without aura in adults. Lasmiditan is a selective and highly potent 5-HT_{IF} receptor agonist (See e.g. Rubio-Beltrán et al., Pharmacol Ther 2018; 186:88-97, and Lasmiditan for the Treatment of Migraine, Capi, M. et al., Expert Opinion Investigational Drugs, (2017), Vol. 26, NO. 2, 227-234). Lasmiditan (COL 144, LY 573144, CAS Registry No. 439239-90-4) can be described chemically as 2,4,6-trifluoro-N-[6-(1-methyl-piperidin-4-ylcarbonyl)-pyridin-2-yl]-benzamide. U.S. Pat. No. 7,423,050 and U.S. Publication No. 20080300407 describe the hemisuccinate salt of 2,4,6-trifluoro-N-[6-(1-methyl-piperidine-4-carbonyl)-pyridin-2-yl]-benzamide having the structural formula:

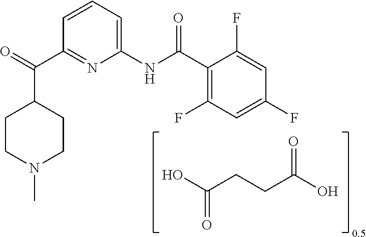

The currently available lasmiditan hemisuccinate solid dosage form, which is a tablet, is acceptable for the treatment purpose. However, this solid dosage form and the potently bitter taste of lasmiditan impose serious compliance problems in patients who are unable or unwilling to take the current solid dosage form of this compound. The solid dosage form is generally difficult for young children and migraine patients experiencing nausea to swallow. Although there are many methods to suppress certain undesired taste of drugs, there is no universal formulation capable of solving this problem due to the unique properties of different drugs. Currently, there is no reported development of a taste masked orally disintegrating tablet (ODT) for lasmiditan.

The taste masked coated lasmiditan orally disintegrating tablet formulation disclosed in this patent application addresses this need. There is a need to develop a palatable orally disintegrating dosage form of lasmiditan to reduce or eliminate its potently bitter taste and other undesirable palatability characteristics, and to avoid the difficulty in swallowing solid dosage forms such as tablets.

The current marketed dosage forms of lasmiditan are immediate release tablets that result in a rapid onset of action (time to symptom relief) of about 2 hours. Treatment of migraines is complicated in that migraine triggers are often not known, and the timing of a migraine is difficult to predict. Convenience of administration of therapy is thus critical to treatment. Most solid oral dosage forms are intended to be swallowed whole and require co-administration of a liquid to facilitate swallowing, reducing convenience of administration. Nausea is a common symptom of migraines, making oral administration of medicaments challenging. If the dosage form requires swallowing liquid or if the dosage form has poor palatability, the migraineur may be reluctant to take the treatment, and/or the medicament may further aggravate the nausea. Further, migraine is one of the most common presenting symptoms in emergency rooms, and patients often have difficulty administering a tablet due to nausea and/or vomiting. Generally, many adults, and especially children, have difficulty swallowing tablets whole even with co-administration of liquid. Dosage forms other than immediate release tablets, that are easier to swallow yet still possess good palatability, are desired in cases where dysphagia is present. The aforementioned problems of delivering migraine therapy orally to adults, and especially pediatric patients, may be resolved through use of orally disintegrating or oro-dispersable tablets if formulation and performance factors necessary for such a tablet can be satisfied. When an orally disintegrating tablet product form can be taken without the need for co-administration with a liquid, it represents a clinically advantageous solution for the migraineur. These ODTs are intended to rapidly disintegrate or disperse, in the small volume of salvia in the mouth, into small particles which are easily swallowed without the need for additional liquid to facilitate swallowing.

Development of ODTs however presents many substantial technical challenges, foremost of which is taste or palatability. Compounds, such as active pharmaceutical ingredients, have taste profiles which vary, and some are highly undesirable. The disagreeable taste of many medicines often requires utilization of taste masking strategies, such as addition of flavors, sweeteners, complexing agents, or other approaches to mask the offensive taste of the medicine. In some instances, there are other negative sensory attributes associated with the medicament, such as trigeminal nerve stimulation, tongue sting, and throat burn that make development of palatable ODTs even more challenging. The challenge of formulating ODT drug products is further complicated when the medicine in question is highly soluble, and a dose greater than a few tens of milligrams is required. Minimizing the negative sensory attributes of high dose, highly soluble drugs, with poor palatability represents a challenge to the extent that the skilled artisan cannot predict whether a clinically suitable ODT product form can successfully be formulated for a highly in-palatable compound.

The technique to use sweetening and flavoring agents to enhance drug taste is one of the most widely used approaches for taste masking, especially in the case of pediatric formulations such as chewable tablets and liquid formulations. However, this approach is not very successful for highly bitter and highly water-soluble drugs. (See for example, Approaches of taste masking. Vishani et al, International Journal of Pharmacy and Integrated Life Sciences, April 2013, Vol I (5). p48-61). Lasmiditan is found to be highly bitter and highly water-soluble drug. In addition, lasmiditan has prolonged bitter taste. Thus, it is expected to be very challenging to make an acceptable palatable ODT dosage form of lasmiditan. However, the present disclosure surprisingly provides pharmaceutical taste masked compositions of lasmiditan in orally disintegrating tablets. The ODT product forms provide compliant dosage forms especially useful in pediatric populations and migraine patients who experience nausea and vomiting when attempting to swallow solid tablets with liquids. The safe and effective treatment of migraine with lasmiditan for patients unable to administer conventional oral tablets would be enabled by the availability of an orally disintegrating tablet which would not require swallowing the tablet. The present disclosure addresses this unmet need for the recently approved migraine treatment lasmiditan.

### SUMMARY

The present disclosure relates to taste masked pharmaceutical compositions of lasmiditan. Specifically, the present disclosure relates to taste masked pharmaceutical compositions comprising a therapeutically effective amount of taste masked lasmiditan particles, comprising lasmiditan or a pharmaceutically acceptable salt thereof, and wherein the particles are coated with one or more taste-masking layers to taste mask the lasmiditan, wherein said taste-masking layer comprises at least one water-insoluble polymer. Preferably the water-insoluble polymer is a reverse enteric coating. Preferably the reverse enteric coating is Kollicoat® Smartseal 30 D. In an embodiment the present disclosure provides a pharmaceutical composition comprising lasmiditan, or a pharmaceutically acceptable salt thereof, and a reverse enteric coating. In an embodiment the present disclosure provides a pharmaceutical composition comprising lasmiditan hemisuccinate and a reverse enteric coating. In an embodiment the present disclosure provides a pharmaceutical composition comprising lasmiditan hemisuccinate and a reverse enteric coating wherein the reverse enteric coating is Kollicoat® Smartseal 30 D, which comprises methyl methacrylate-di(ethyl)aminoethyl methacrylate copolymer. In an embodiment the present disclosure provides a pharmaceutical composition comprising lasmiditan hemisuccinate, wherein the lasmiditan comprises granulated particles having a size range of about 50 to about 275 microns, and a reverse enteric coating wherein the reverse enteric coating is Kollicoat® Smartseal 30 D. In an embodiment the present disclosure provides a pharmaceutical composition comprising lasmiditan hemisuccinate, wherein the lasmiditan comprises granulated particles having a size range of about 50 to about 275 microns, wherein the lasmiditan to be coated further comprises talc, and a reverse enteric coating wherein the reverse enteric coating is Kollicoat® Smartseal 30 D, wherein the final coated particles have a size range between about 75 and about 300 microns.

In an embodiment the present disclosure provides a pharmaceutical composition comprising lasmiditan hemisuccinate, wherein the lasmiditan comprises granulated particles having a size range of about 50 to about 275 microns, wherein the composition further comprises about 20-40% coat level upon coating with Kollicoat® Smartseal 30 D.

In an embodiment the present disclosure provides a pharmaceutical composition comprising lasmiditan hemisuccinate, wherein the lasmiditan comprises granulated particles having a size range of about 50 to about 275 microns, wherein the composition further comprises about 37% coat level upon coating with Kollicoat® Smartseal 30 D.

In an embodiment the present disclosure provides a pharmaceutical composition comprising lasmiditan hemisuccinate, wherein the lasmiditan comprises granulated particles having a size range of about 50 to about 275 microns, wherein the lasmiditan to be coated further comprises talc, and a reverse enteric coating wherein the reverse enteric coating is Kollicoat® Smartseal 30 D, wherein the final coated particles have a size range between about 75 and about 300 microns which further comprises:
(i) about 55.5% w/w of lasmiditan hemisuccinate,
(ii) about 6.0% w/w of Hypromellose (HPMC),
(iii) about 0.15% w/w of Sodium Lauryl Sulfate,
(iv) about 2.8% w/w of Triethyl Citrate,
(v) about 18.6% w/w of Kollicoat® Smartseal 30 D, and
(vi) about 16.9% w/w of Talc.

In an embodiment the present disclosure provides a pharmaceutical composition comprising lasmiditan hemisuccinate, wherein the lasmiditan comprises granulated particles having a size range of about 50 to about 275 microns, wherein the lasmiditan to be coated further comprises talc and a reverse enteric coating wherein the reverse enteric coating is Kollicoat® Smartseal 30 D, wherein the final coated particles have a size range between about 75 and about 300 microns, and a disintegrant and a lubricant.

In an embodiment the present disclosure provides a pharmaceutical composition comprising lasmiditan hemisuccinate, wherein the lasmiditan comprises granulated particles having a size range of about 50 to about 275 microns, wherein the lasmiditan to be coated further comprises talc and a reverse enteric coating, wherein the reverse enteric coating is Kollicoat® Smartseal 30 D, wherein the final coated particles have a size range between about 75 and about 300 microns, wherein the composition further comprises Talc, Pharmaburst® 500, and sodium stearyl fumarate.

In an embodiment the present disclosure provides a pharmaceutical composition comprising lasmiditan hemisuccinate, wherein the lasmiditan comprises granulated particles having a size range of about 50 to about 275 microns, wherein the lasmiditan to be coated further comprises talc and a reverse enteric coating, wherein the reverse enteric coating is Kollicoat® Smartseal 30 D, wherein the final coated particles have a size range between about 75 and about 300 microns, wherein the composition further comprises Talc, Pharmaburst® 500, and Sodium Stearyl Fumarate, and wherein the composition further comprises a sweetener and a flavoring agent.

In an embodiment the present disclosure provides a pharmaceutical composition comprising lasmiditan hemisuccinate, wherein the lasmiditan comprises granulated particles having a size range of about 50 to about 275 microns, wherein the lasmiditan to be coated further comprises talc and a reverse enteric coating, wherein the reverse enteric coating is Kollicoat® Smartseal 30 D, wherein the final coated particles have a size range between about 75 and about 300 microns, wherein the composition further comprises Talc, Pharmaburst® 500, and Sodium Stearyl Fumarate, and wherein the composition further comprises Aspartame and Cherry berry flavoring agent.

In an embodiment the present disclosure provides a pharmaceutical composition comprising lasmiditan hemisuccinate, wherein the lasmiditan comprises granulated particles having a size range of about 50 to about 275 microns, wherein the lasmiditan to be coated further comprises talc and a reverse enteric coating, wherein the reverse enteric coating is Kollicoat® Smartseal 30 D, wherein the final coated particles have a size range between about 75 and about 300 microns, wherein the composition further comprises Talc, Pharmaburst® 500, and Sodium Stearyl Fumarate, and wherein the composition further comprises Aspartame and Cherry berry flavoring agent, wherein the composition further comprises:
(i) about 40.2% w/w of Kollicoat® Smartseal 30 D Coated lasmiditan hemisuccinate (37% coat level),
(ii) about 0.80% w/w of Talc,
(iii) about 54.0% w/w of Pharmaburst® 500,
(iv) about 2.0% w/w of Sodium Stearyl Fumarate,
(v) about 1.0% w/w of Cherry berry flavoring, and
(vi) about 2.0% w/w of Aspartame.

In an embodiment the present disclosure provides a pharmaceutical composition comprising lasmiditan hemisuccinate, wherein the lasmiditan comprises granulated particles having a size range of about 50 to about 275 microns, wherein the lasmiditan to be coated further comprises talc and a reverse enteric coating, wherein the reverse enteric coating is Kollicoat® Smartseal 30 D, wherein the final coated particles have a size range between about 75 and about 300 microns, wherein the composition further comprises Talc, Pharmaburst® 500, and Sodium Stearyl Fumarate, and wherein the composition further comprises Aspartame and Cherry berry flavoring agent, wherein the composition further comprises:
about 37% to 46% w/w of Kollicoat® Smartseal 30 D Coated Lasmiditan Hemisuccinate,
about 47% to 58% w/w of Pharmaburst® 500,
about 3.9% to 4.9% w/w of aspartame/Cherry berry flavoring blend (Aspartame about 68% to Cherry Berry Flavor about 32% w/w); and
about 1.3% to 1.7% w/w of Sodium Stearyl Fumarate.

In an embodiment the present disclosure provides a pharmaceutical composition according to any of the above embodiments, wherein the composition further comprises a dosage of lasmiditan from about 25 mg to about 200 mg.

In an embodiment the present disclosure provides a pharmaceutical composition according to any of the above embodiments, wherein the composition further comprises a dosage of lasmiditan from about 25 mg to about 100 mg.

In an embodiment the present disclosure provides a pharmaceutical composition according to any of the above embodiments, wherein the composition further comprises a dosage of lasmiditan of about 25 mg.

In an embodiment the present disclosure provides a pharmaceutical composition according to any of the above embodiments, wherein the composition further comprises a dosage of lasmiditan of about 50 mg.

In an embodiment the present disclosure provides a pharmaceutical composition according to any of the above embodiments, wherein the composition further comprises a dosage of lasmiditan of about 75 mg.

In an embodiment the present disclosure provides a pharmaceutical composition according to any of the above embodiments, wherein the composition further comprises a dosage of lasmiditan of about 100 mg.

In an embodiment the present disclosure provides a pharmaceutical composition according to any of the above embodiments, wherein the composition further comprises a dosage of lasmiditan of about 150 mg.

In an embodiment the present disclosure provides a pharmaceutical composition according to any of the above embodiments, wherein the composition further comprises a dosage of lasmiditan of about 200 mg.

In an embodiment the present disclosure provides a pharmaceutical composition according to any of the above embodiments, wherein the composition further comprises an orally disintegrating tablet.

In an embodiment the present disclosure provides a pharmaceutical composition according to any of the above embodiments, wherein the composition further comprises an orally disintegrating tablet wherein the tablet further comprises a unit dosage of 25 mg.

In an embodiment the present disclosure provides a pharmaceutical composition according to any of the above embodiments, wherein the composition further comprises an orally disintegrating tablet wherein the tablet further comprises a unit dosage of 50 mg.

In an embodiment the present disclosure provides a pharmaceutical composition according to any of the above embodiments, wherein the composition further comprises an orally disintegrating tablet wherein the tablet further comprises a unit dosage of 100 mg.

In an embodiment the present disclosure provides a method of treating migraine in a patient comprising administering to a patient in need of such treatment an effective amount of a composition according to any of the above embodiments of lasmiditan compositions.

In an embodiment the present disclosure provides a composition according to any the above embodiments of lasmiditan compositions for use in therapy.

In an embodiment the present disclosure provides a composition according to any the above embodiments of lasmiditan compositions for use in the treatment of migraine.

The present disclosure also relates to an immediate release (IR) orally disintegrating tablet (ODT) comprising a therapeutically effective amount of lasmiditan particles wherein each particle comprises 2,4,6-trifluoro-N-[6-(1-methyl-piperidin-4-ylcarbonyl)-pyridin-2-yl]-benzamide, or a pharmaceutically acceptable salt thereof, coated with one or more taste-masking layers, wherein the taste-masking layer comprises a water-insoluble polymer. The present disclosure provides a palatable pharmaceutical composition in the form of taste-masked 2,4,6-trifluoro-N-[6-(1-methylpiperidine-4-carbonyl)-2-pyridyl]benzamide hemisuccinate and orally disintegrating tablets comprising the same.

The present disclosure further provides a compressed orally disintegrating tablet comprising a disintegrant and a plurality of units comprising:
i) a plurality of particles comprising a therapeutically effective amount of lasmiditan or a pharmaceutically acceptable salt thereof;
ii) a reverse enteric coating over the particles comprising a reverse enteric polymer in an amount of 20% to 40% coat level;
wherein the disintegrant and the plurality of units are compressed to an orally disintegrating tablet having a friability of 1% or less when 6 kN to 50 kN of a compression force is applied during manufacturing of the tablet.

The present disclosure further provides a process of manufacturing the orally disintegrating tablet of any of the above embodiments comprising:
a) generating a plurality of particles comprising a therapeutically effective amount of lasmiditan, or a pharmaceutically acceptable salt thereof;
b) applying a coating comprising a reverse enteric polymer to the particles of step (a) thereby obtaining a plurality of units;
c) mixing the plurality of units of step (b) with at least one tablet excipient comprising a disintegrant thereby obtaining a blend;
d) mixing the blend of step (c) with a flavor and a sweetener to make a taste masked blend;
e) mixing the taste masked blend with a dry lubricant; and
f) compressing the blend of step (e) thereby obtaining the compressed orally disintegrating tablet.

The present disclosure also provides methods of making the taste masked and ODT compositions and methods of using the present compositions for treating a patient subject to migraine attacks. The taste-masked orally disintegrating tablets of this disclosure will significantly reduce the potently bitter taste of lasmiditan and enable administration of this product form to migraine patients, in particular pediatric patients, and those suffering from nausea due to migraine attacks.

The present disclosure relates to a solid pharmaceutical composition comprising taste masked lasmiditan, or a pharmaceutically acceptable salt thereof, incorporated into an orally disintegrating tablet (ODT), preferably wherein the tablet disintegrates within about 30 seconds. The present disclosure further provides ODTs possessing desired mechanical strength and desired in-vitro release profiles comprising taste masked lasmiditan, along with one or more pharmaceutically acceptable excipients.

### DETAILED DESCRIPTION

The following description includes information useful in understanding the present disclosure.

### DEFINITONS

As used above and throughout the disclosure the following terms, unless otherwise indicated, shall be understood to have the following meanings: The term “drug”, “active”, “active ingredient”, or “active pharmaceutical ingredient” as used herein includes any pharmaceutically acceptable and therapeutically effective compound or pharmaceutically acceptable salt thereof. A preferred compound of the present disclosure is 2,4,6-trifluoro-N-[6-(1-methyl-piperidine-4-carbonyl)-pyridin-2-yl]-benzamide. A preferred compound of the present disclosure is 2,4,6-trifluoro-N-[6-(1-methyl-piperidine-4-carbonyl)-pyridin-2-yl]-benzamide hemisuccinate. A preferred compound of the present disclosure is 2,4,6-trifluoro-N-[6-(1-methyl-piperidine-4-carbonyl)-pyridin-2-yl]-benzamide hemisuccinate in solid Form A. A preferred compound of the present disclosure is 2,4,6-trifluoro-N-[6-(1-methyl-piperidine-4-carbonyl)-pyridin-2-yl]-benzamide hemisuccinate in solid Form D.

Methods of preparing lasmiditan and salts and certain polymorphic forms, formulations, and dosage forms thereof, are known to the skilled artisan, and are described for example in WO 2003/084949, WO 2011/123654, WO 2018/106657, and WO 2021/007155. As used herein, useful forms of lasmiditan (also referred to as LY573144) include pharmaceutically acceptable salts thereof, including but not limited to 2,4,6-trifluoro-N-[6-(1-methyl-piperidin-4-ylcarbonyl)-pyridin-2-yl]-benzamide mono-hydrochloride salt, and 2,4,6-trifluoro-N-[6-(1-methyl-piperidine-4-carbonyl)-pyridin-2-yl]-benzamide hemi-succinate salt. A synthetic route for the preparation of the hemi-succinate salt of 2,4,6-trifluoro-N-[6-(1-methyl-piperidine-4-carbonyl)-pyridin-2-yl]-benzamide has been disclosed previously (see for example WO 2021/007155).

“Pharmaceutically acceptable salts” or “a pharmaceutically acceptable salt” refers to the relatively non-toxic, inorganic and organic salt or salts of the compound of the present invention. It will be understood by the skilled artisan that compounds of the present invention are capable of forming salts. The compounds of the present invention contain basic heterocycles, and accordingly react with any of a number of inorganic and organic acids to form pharmaceutically acceptable acid addition salts. Such pharmaceutically acceptable acid addition salts and common methodology for preparing them are well known in the art. See, e.g., P. Stahl, et al., HANDBOOK OF PHARMACEUTICAL SALTS: PROPERTIES, SELECTION AND USE, (VCHA/Wiley-VCH, 2008); S. M. Berge, et al., “Pharmaceutical Salts”, Journal of Pharmaceutical Sciences, Vol 66, No. 1, January 1977.

As used herein, the term “reverse enteric coating” means, in the broadest meaning reverse enteric polymers used as a barrier coat. As used herein, the term “reverse enteric coating” refers to a coating comprising a “reverse enteric polymer” which refers to pH sensitive polymers, which are insoluble at pH values greater than those found in the stomach i.e. at pH values greater than 5.0, while being soluble at acidic pH values. Suitable reverse enteric polymers are thus insoluble in the oral cavity and soluble in the stomach. In some embodiments, the reverse enteric polymer is a copolymer of hydrophobic monomers and/or basic monomers; non-limiting examples of such reverse enteric polymers are described in U.S. Patent Application No. 2006/0134054. In certain embodiments, the monomer is an acrylic or a methacrylic acid ester comprising, but not limited to, methyl (meth)acrylate, benzyl (meth)acrylate, dodecyl (meth)acrylate, octyl (meth)acrylate, cyclohexyl (meth)acrylate, phenyl (meth)acrylate, tertiary butyl (meth)acrylate, butyl (meth)acrylate, ethyl hexyl (meth)acrylate, propyl (meth)acrylate, or combinations thereof. Each possibility represents a separate embodiment. In other embodiments, the monomer is a substituted acrylic or a methacrylic acid ester comprising, but not limited to, dimethyl amino ethyl (meth)acrylate, diethyl amino ethyl (meth)acrylate, piperidine ethyl (meth)acrylate, tertbutyl amino ethyl (meth)acrylate, EUDRAGIT® E 100, Eudragit® EPO, or combinations thereof. Each possibility represents a separate embodiment. Preferred reverse enteric coatings of the present embodiments include Kollicoat® Smartseal 30 D or Kollicoat® Smartseal 100 P (The BASF PRD number (product number) is listed as 30492630 for Kollicoat® Smartseal 30 D, and 30585559 for Kollicoat® Smartseal 100 P). Kollicoat® Smartseal 100 P coating can be applied using the 100 P (Powder) grade using an organic solvent system (e.g. alcohol or acetone). A particularly preferred reverse enteric coatings of the present embodiments is Kollicoat® Smartseal 30 D (30% Dispersion). The term “unit” as used herein, refers to applying a coating comprising a reverse enteric polymer to granulated particles of lasmiditan, or a pharmaceutically acceptable salt thereof, thereby obtaining a plurality of units of coated API.

As used herein, the term “patient” refers to a human. As used herein, the terms “treatment”, “treating”, or “mitigating” are intended to refer to all processes wherein there may be a slowing, interrupting, arresting, controlling, or stopping of the progression of an existing disorder and/or a reduction in symptoms thereof, but does not necessarily indicate a total elimination of all symptoms. As used herein, the term “effective amount” of lasmiditan, refers to an amount, that is a dosage, which is effective in treating migraine in a patient. A preferred “effective amount” is determined as an amount that can treat or eliminate the signs and symptoms of migraine attack in the patient, as compared to the patient when untreated. Preferred amounts of lasmiditan include the range from 25-200 mg, and unit dosages of 25 mg, 50 mg, 100 mg, and 200 mg.

A “dose” refers to a predetermined quantity of lasmiditan calculated to produce the desired therapeutic effect in a patient. As used herein “mg” refers to milligram. As used herein, doses described in mg, refer to the active pharmaceutical ingredient lasmiditan as free-base equivalent by mass, for instance a “100 mg” dose, refers to 100 mg of the active pharmaceutical ingredient lasmiditan as free-base equivalent. As used herein, a given dose may be interpreted to describe doses of about the indicated amount, in that doses which are up to 10 percent higher or lower than the indicated dose are likewise contemplated to provide useful regimens in a manner similar to the indicated dose. A pharmaceutical composition of lasmiditan of the present disclosure can be provided in bulk or in dosage unit form. It is especially advantageous to formulate pharmaceutical compositions of lasmiditan in dosage unit form for ease of administration and uniformity of dosage. The term “dosage unit form” as used herein refers to physically discrete units suitable as unitary dosages for the subject to be treated; each unit containing a predetermined quantity of active compound lasmiditan calculated to produce the desired therapeutic effect in association with the required pharmaceutical carrier. A dosage unit form can be, e.g., an orally disintegrating tablet comprising a preferred dose of lasmiditan, such as 25 mg, 50 mg, 100 mg, and 200 mg

In embodiments, the disclosure provides a pharmaceutical composition comprising an amount of lasmiditan in ODT form as described herein wherein the amount is from 25 mg to 200 mg per dose. In embodiments, the disclosure provides a pharmaceutical composition comprising an amount of lasmiditan in ODT form as described herein wherein the amount is 25 mg, 50 mg, 75 mg, 100 mg, 150 mg or 200 mg per dose. The forgoing doses are based on an adult human of average weight, and/or the smaller doses would be acceptable for individuals of lighter weight, for example the elderly or children.

In embodiments of the present disclosure the patient is a human who has been diagnosed as having a condition or disorder in need of treatment with a pharmaceutical composition described herein. In some embodiments, a patient is a human that is characterized as being at risk of a condition or disorder for which administration with a pharmaceutical composition described herein is indicated. In those instances where the disorders which can be treated by the methods of the present invention are known by established and accepted classifications, such as migraine, episodic headache, chronic headache, chronic cluster headaches, and/or episodic cluster headaches, their classifications can be found in various sources. For example, at present, the fourth edition of the Diagnostic and Statistical Manual of Mental Disorders (DSM-IVTM) (1994, American Psychiatric Association, Washington, D.C.), provides a diagnostic tool for identifying many of the disorders described herein. Also, the International Classification of Diseases, Tenth Revision (ICD-10), provides classifications for many of the disorders described herein. The skilled artisan will recognize that there are alternative nomenclatures, nosologies, and classification systems for disorders described herein, including those as described in the DSM-IV and ICD-10, and that terminology and classification systems evolve with medical scientific progress. Migraine patients can further be diagnosed with migraine, with or without aura (1.1 and 1.2), as defined by International Headache Society (IHS) International Classification of Headache Disorders, 3rd edition, (ICHD-3) beta version (The International Classification of Headache Disorders, 3rd edition (beta version), Cephalalgia 2013; 33: 629-808). In some embodiments, the human patient has been diagnosed with episodic migraine prior to receiving administration of lasmiditan to treat migraine. In some embodiments, the human patient has been diagnosed with chronic migraine prior to receiving lasmiditan. In some embodiments, the human patient experiences auras with their migraine headaches. In some embodiments, the human patient does not experience auras with their migraine headaches.

As used herein “migraine” includes but is not limited to migraine attacks. As used herein “migraine attack” refers to the following description. Symptoms may overlap within various phases of a migraine attack and not all patients experience the same clinical manifestations. In the prodrome phase, the majority of patients have premonitory symptoms that may precede the headache phase by up to 72 hours. These include changes in mood and activity, irritability, fatigue, food cravings, repetitive yawning, stiff neck, and phonophobia. These symptoms may endure well into the aura, headache, and even postdrome phases. Some patients experience an aura phase, wherein about one-third of patients experience transient neurological deficits during attacks. The ICHD-3 defines aura as 1 or more transient, fully reversible neurological deficits, of which at least 1 has to have a unilateral localization, that develops over 5 minutes or more, and of which each deficit lasts between 5 and 60 minutes. While a visual aura, which may show positive (fortification spectra), negative (scotoma), or both phenomena, is found in over 90% of the cases, and the most common deficit, sensory, motor, speech, brain stem, and retinal aura symptoms may also occur. A transient wave of neuronal depolarization of the cortex is believed to be the pathophysiological brain mechanism underlying the clinical phenomenon of migraine aura. In the headache phase, headache attacks which may last 4 to 72 hours are accompanied by nausea, photophobia and phonophobia, or both. The headache is characterized as unilateral, pulsating, of moderate or severe intensity, and aggravated by physical activity; two of these characteristics suffice to fulfill the diagnostic criteria. In the postdrome phase, characteristic symptoms reflect those observed during the premonitory phase. Typical postdrome symptoms include tiredness, difficulties in concentrating, and neck stiffness. It remains unclear whether these symptoms initiate in the premonitory phase and persist throughout the headache phase into the postdrome phase, if they may also initiate during the headache phase, or even appear after the headache phase has ended.

A “migraine headache” as used herein refers to headache, with or without aura, of ≥30 minutes duration, with both of the following required features (A and B): A) at least 2 of the following headache characteristics: 1) unilateral location, 2) pulsating quality, 3) moderate or severe pain intensity, and 4) aggravation by or causing avoidance of routine physical activity; AND B) during headache at least one of the following: a) nausea and/or vomiting, and/or b) photophobia and phonophobia. A “probable migraine headache” as used herein refers to a headache of greater than 30 minutes duration, with or without aura, but missing one of the migraine features in the International Headache Society ICHD-3 definition.

The abbreviations listed below when used herein are defined as follows: “CAS No.” means Chemical Abstracts Registry number. “hr” or “h” means hour or hours. “NMT” means not more than. “RT” means room temperature/ambient temperature. “sec” means second or seconds as a unit of time. “w/w” means weight to weight in a ratio.

Compositions, processes, product forms and uses of the present disclosure are further described in terms of certain preferred embodiments including the preparation of reverse enteric coated lasmiditan and orally disintegrating tablets comprising the coated lasmiditan. A palatable, taste-masked commercially viable orally disintegrating tablet of lasmiditan hemisuccinate was developed for introduction into a bioequivalence study (LAIA). Lasmiditan in this disclosure refers to 2,4,6-trifluoro-N-[6-(1-methylpiperidine-4-carbonyl)-2-pyridyl]benzamide per se. The particular salt used in this disclosure is the hemisuccinate salt, however other salts such as the hydrochloride or other suitable salts are within the embodiments of the present disclosure.

### Challenges for the Preparation of Orally Disintegrating Tablets of Lasmiditan:

Orally disintegrating tablets (ODTs) are solid oral dosage forms that dissolve rapidly in the saliva of oral cavity allowing the medicine to be easily swallowed without water. This is beneficial in patients with dysphagia (e.g. pediatric), in diseases where symptoms may preclude consuming liquid (nausea), and where convenience of administration is desirable (migraine). ODTs however present challenges in formulation development beyond the typical critical quality attributes of immediate release tablets (e.g. purity, potency). ODTs are also required to be palatable to the patient to ensure adherence; with rapid oral disintegration and pleasant taste being paramount. The present disclosure addresses the challenges and provides novel solutions for an ODT product form for REYVOW® (lasmiditan) for pediatric and/or adult populations.

Lasmiditan is highly soluble (dissolves readily in the mouth) but extremely bitter in taste, and has other negative sensory attributes, that preclude conventional ODT development. For lasmiditan the solubility is 35 to 9.8 mg/mL at pH 5 to 6.8, which is roughly the pH range of the oral cavity. The efficacious dose is from 25 to 200 mg depending on patient weight or other factors. A taste study using trained taste panelists and crushed 50 mg (e.g. 2×50 mg) tablets Lasmiditan immediate release tablets showed that lasmiditan has very poor palatability attributes. Extreme bitterness, mouth numbing, and other negative sensory attributes are present and persist for 30 minutes.

TABLE 1
Lasmiditan flavor profile as a function of time.

| Flavor | | | | | | | | | |
|---|---|---|---|---|---|---|---|---|---|
| Profile | Intensity of sensory characteristic at time post dose expectoration | | | | | | | | |
| Attribute / | for 100 mg Lasmiditan dose* | | | | | | | | |
| Sensory | | 1 | 3 | 5 | 10 | 15 | 20 | 25 | 30 |
| Characteristic | Initial | min | min | min | min | min | min | min | min |
| Bitterness | 3 | 3 | 3 | 2.5 | 2.5 | 2.5 | 2 | 2 | 1.5 |
| Chalky | 1.5 | 1 | 0 | 0 | 0 | 0 | 0 | 0 | 0 |
| Aromatic | | | | | | | | | |
| Sour | 1.5 | 1 | 0 | 0 | 0 | 0 | 0 | 0 | 0 |
| Polyethylene- | 1.5 | 1.5 | 1 | 0 | 0 | 0 | 0 | 0 | 0 |
| like aromatic | | | | | | | | | |
| Chalky | 1.5 | 1 | 0 | 0 | 0 | 0 | 0 | 0 | 0 |
| mouthfeel | | | | | | | | | |
| Tannin | 1.5 | 1.5 | 1 | 0 | 0 | 0 | 0 | 0 | 0 |
| mouthfeel | | | | | | | | | |
| Tongue | 1 | 1 | 1.5 | 1.5 | 1 to 1.5 | 1 | 0.5 | 0.5 | 0.5 |
| sting | | | | | | | | | |
| Metallic | 1.5 | 1.5 | 1.5 | 1.5 | 1.5 | 1 | 1 | 0.5 | 0 |
| Aromatic | | | | | | | | | |
| Throat burn | 0 | 1 | 1 | 1 | 1 | 1 | 0 | 0 | 0 |
| Mouth Numbing | 0 | 0 | 1 | 1.5 | 1.5 | 1.5 | 1 | 1 | 1 |

*The intensity scale ranges from 0 (no intensity/non-detectable) to 3 (highly intense). Aversive sensory characteristics above a slight intensity (>1) are clearly perceptible to patients and are often found to be unacceptable.

Approaches to limit the negative sensory attributes of particularly poorly tasting medicine may include applying a barrier coating to the drug substance to prevent dissolution in the oral cavity. An approach is to use an insoluble film containing soluble pore forming agents such as cellulose acetate with polyethylene glycol, or ethyl cellulose with hypromellose. The challenge with this approach is to balance the amount of soluble pore former with insoluble polymer to ensure the drug is properly taste masked while still rapidly releasing in the gastrointestinal tract to ensure adequately rapid absorption and onset of action; particularly critical for migraineurs. Reverse enteric polymers have also been used as a barrier coat. These polymers are designed to be insoluble at the pH of saliva but rapidly dissolve at the pH of the stomach. Reverse enteric polymers have also been demonstrated as pore formers in otherwise insoluble films.

U.S. Pat. No. 5,489,436 recites an example of the use of the reverse enteric polymer Eudragit® 100E as the pore former in insoluble cellulose ester films. This approach has the limitation of requiring effort to define the optimal amount of pH sensitive pore former to include in the film such to achieve good taste masking performance, while not compromising release in-vivo due to the insoluble film coating. In the case of medicaments for relief of migraine symptoms, any delay in drug release may result in a delay of absorption and a delay in pharmacodynamic effect. An ideal taste masking film would have almost no release in the mouth but instantaneous and complete drug release in the GI tract equivalent to that of a conventional immediate release tablet.

Orally disintegrating tablets must also meet other constraints, such as rapid disintegration. The FDA guidance states is that tablets must disintegrate in not more than 30 seconds using conventional USP <711> disintegration testing. The FDA also generally recommends that the weight of the ODT tablet not exceed 500 mg; however, if a tablet intended for use as an ODT weighs more than 500 mg, its ability to perform effectively as an ODT should be justified based on product performance. Finally, ODTs must be hard and robust enough such that the integrity and elegance of the tablet is not compromised during manufacturing, packaging, or handling by the patient. Achieving these requirements for doses greater than a few tens of mg is difficult as many of the desired attributes such as tablet hardness and rapid disintegration are at odds; meaning soft tablets disintegrate rapidly, but are difficult to handle, and vice versa, hard tablets are easy to handle, but have slow disintegration.

### Compositions and Orally Disintegrating Tablet Formulations and Product Forms of the Present Disclosure

The present disclosure describes embodiments of an orally disintegrating tablet (ODT) form of lasmiditan, referred to herein as “lasmiditan ODT”, useful for the acute treatment of migraine in patients with and without aura. The following preparations of ODT tablets of lasmiditan further illustrate the invention and represent typical preparations. The reagents and starting materials are readily available or may be readily synthesized by one of ordinary skill in the art. It should be understood that the Preparations and Examples are set forth by way of illustration, and that various modifications may be made by one of ordinary skill in the art.

### Preparation of the Taste Masked Drug Substance

The raw medicament lasmiditan hemisuccinate is preferably prepared in the size range of about 50 to no more than 275 μm to be suitable for small particle coating. It is recognized that coating of particles less than around 50 μm, referred to as fines here, is not generally practical or feasible. The high surface area of fines requires high levels of coating for taste masking and/or may require a granulation step to tie up fines. Furthermore, it is recognized that the persistence of fine particles should be minimized during coating as the presence of fine particles may lead to poor final coating and compromise taste masking effectiveness. It is also recognized that particles greater in size than about 300 μm are not desired in an ODT as theses can lead to a gritty mouthfeel in the final product.

Small particles as defined herein are those particles in the general range of d10 of around 50 μm, and d90 not to exceed about 275 μm, and coating may be performed in a several ways, such as coacervation and fluid bed coating. A common way is using Wurster style fluid bed coaters as this process generally provides for an efficient coating process and is a well understood process. In an embodiment of the present disclosure, lasmiditan drug substance is coated using Wurster style fluid bed coating.

Particle size determination is known the skilled artisan and can employ well known methods. Materials and Equipment used can include Malvern Mastersizer 3000 particle size analyzer with Aero S Module, a Dispersing System: Micro tray standard venturi disperser, and current windows software or equivalent with Malvern Mastersizer 3000 software (Version 3.0 or equivalent). Measurements are conducted by standard procedures (see for example Malvern Mastersizer 3000 Laser Diffraction Particle Size Distribution Analyzer Operation, Calibration, and Maintenance, current version of PPD SOP IO 237, and Light Diffraction Measurement of Particle Size, current version of USP) to calculate the average for d10, d50 and d90 of the three test article preparations.

In accordance with preferred embodiments of the present disclosure, the raw medicament is first granulated/sub-coated with HPMC E5 prior to application of the reverse enteric co-polymer top-coat. A surfactant may also be included in the coating solution to ensure good wetting of the coating solution onto the particle. Sodium lauryl sulfate is a preferred surfactant. A sub-coat/granulation step serves to both bind fine particles into a granule, as well as provide greater particle core integrity to avoid particle attrition during coating, both serving to improve yield and quality of the taste mask coating.

In an aspect the present invention is directed to the discovery of a reverse enteric coated lasmiditan composition, and incorporation into an ODT, which achieves a balance between in-vitro taste masking, in-vitro dissolution (supporting rapid rate of bioavailability), rapid disintegration time, and adequate tablet hardness.

The present disclosure provides an ODT comprised of lasmiditan hemisuccinate drug substance coated with an effective amount of a polymer coating for taste masking, preferably a reverse enteric coating. Reverse enteric coatings are defined herein as polymer or co-polymer coatings which are not soluble at pH's greater than that which is typical in the mouth (typically about pH 6 to 7) but are soluble in the fluids of the stomach having lower pH's, for example pH 1.0 to about 3.5-5.0. Preferably compositions of the present disclosure comprise a coating of the reverse enteric methyl methacrylate-di(ethyl)aminoethyl methacrylate copolymer, Kollicoat® Smartseal 30 D (commercially available from BASF). Prior to application of the co-polymer coating, the neat drug substance is preferably granulated using an inert polymer, such as HPC, or HPMC, and preferably HPMC E5. Talc may be added to any coating to facilitate processing. The particle size of the starting API is preferably in the size range of approximately 50 to 275 microns to facilitate particle coating while keeping the coated particles to a size that will not feel gritty in the mouth in the final dosage form. The resulting coated particle may also be dusted with an anti-caking agent such as colloidal silicon dioxide or talc, preferably talc, to minimize caking upon storage.

The coating process is made easier with the incorporation of talc in the coating suspensions to minimize tackiness of the particles during coating. High tackiness during processing leads to increased particle-particle sticking and agglomeration. Particle agglomeration decreases efficiency of coating leading to erratic drug release profiles from batch to batch. In addition, if tackiness occurs extensively during processing, the granulation will ball up into solid masses (or agglomerates) greater than 300 μm which would have a gritty feel in the mouth. The final coated particle is desired to be in the approximate size range of 75 to 300 μm to facilitate processing into an ODT, while avoiding a gritty feel in the mouth in the final product. The following unit formula can be used in manufacturing ODT lasmiditan tablets as follows for 25 mg, 50 mg, and 100 mg doses:

TABLE 2

| | Quantity (mg/tablet) | | | |
|---|---|---|---|---|
| Ingredient | 25 mg | 50 mg | 100 mg | w/w % |
| Active | | | | |
| Kollicoat ® Smartseal | 52.050 | 104.100 | 208.200 | 41.64% |
| 30 D Coated | | | | |
| lasmiditan | | | | |
| hemisuccinate^{A} | | | | |
| As lasmiditan | 25.000 | 50.000 | 100.000 | 20.00% |
| Freebase | | | | |
| Other Ingredients | | | | |
| Pharmaburst ® 500^{B} | 65.575 | 131.150 | 262.300 | 52.46% |
| Aspartame | 3.750 | 7.500 | 15.000 | 3.000% |
| N-C Cherry Berry | 1.750 | 3.500 | 7.000 | 1.400% |
| Flavor, Art (FONA | | | | |
| International product | | | | |
| code 825.0062U) | | | | |
| Sodium Stearyl | 1.875 | 3.750 | 7.500 | 1.500% |
| Fumarate | | | | |
| Total Weight (mg) | 125.0 | 250.0 | 500.0 | 100% |

^{A}The amount of drug substance (drug product intermediate, coated API) is based on the Assay of the active ingredient.
^{B}The amount of Pharmaburst ® 500 is adjusted accordingly to maintain the theoretical tablet weight.

The taste masked coated lasmiditan is preferably directly compressed with excipients suitable to prepare ODTs. The excipients may be any of those commonly used in the production of ODTs such as polyols (mannitol, sorbitol), fillers (starches, microcrystalline cellulose), lubricants (sodium stearyl fumarate, magnesium stearate, talc), flow aides (colloidal silicon dioxide), disintegrants (crospovidone, sodium croscarmellose). Preferably, a co-processed excipient designed for ODTs, such as Pharmaburst® 500 (commercially available from SPI Pharma), may be used to simplify processing and optimize tablet properties. Flavors (mint, cherry berry, peppermint) and sweeteners (aspartame, sucralose, neotame) may also be added as is common in ODT preparations. A preferred flavor is FONA N-C Cherry Berry Flavor ART #825.0062U. A preferred sweetener is aspartame. Alternative flavors are N-C Cherry Flavor ART-825.0597U, Bubblegum Flavor ART-815.0084U, N-C Strawberry Flavor ART-915.0435U, Fonatech Mango Flavor NAT WONF-870.0235U, Juicy Orange Flavor NAT WONF-884.0107U. The tablet is compressed to a solid fraction that is high enough to ensure low tablet friability (less than 1%) in downstream processing, while also maintaining an in-vitro disintegration time of not more than 30 seconds.

### BRIEF DESCRIPTION OF THE DRAWINGS

FIG. 1: Process Flow Chart for Lasmiditan ODT Drug Product

FIG. 2: Process Flow Diagram for Lasmiditan ODT Drug Product Intermediate

FIG. 3: Taste Profiling of Lasmiditan ODT embodiments using a Flavor Profile Method

FIG. 4: Illustrative examples of lasmiditan hemisuccinate orally disintegrating tablets

### EXAMPLES

The following examples are offered to illustrate, but not to limit, the claimed inventions. The results of the following methods and procedures demonstrate that the exemplified compositions, formulations, and tablets of the present disclosure provide useful drug product intermediates and drug product forms for lasmiditan for orally disintegrating tablets, and therefore may be used for treating migraine and or headache disorders.

### Example 1

### General Procedure to Coat lasmiditan

The following procedure describes how to coat 1.2 kg of lasmiditan hemisuccinate of a particle size of d10=55.0 μm, d50=117.9 μm, and d90=220.9 μm, or similar. Charge the fluid bed coater as described in Table 3 with lasmiditan hemisuccinate. There are many vendors who supply fluid bed coaters capable of Wurster coating and the equipment set-up may differ between vendors, particularly with respect to nozzle type and fluidization parameters. The examples cited here are for one particular style of fluid bed coater, but it is understood that other fluid bed coaters may be used to achieve similar results.

TABLE 3
Equipment description for fluid bed coating.

| Equipment | Description |
|---|---|
| Fluid bed coater | CPI Model 600 |
| Chamber | 6″ short |
| Partition | 3″ × 6″ × 0.5″ |
| Nozzle | CPI #6 generation 2 with 1 extension |
| Fluidizing plate | W6-10-1 with 325 mesh screen |

Prepare the sub-coat/granulating solution of HPMC E5 and SLS in purified water as shown in Table 4.

TABLE 4
Sub-coat/granulating fluid composition.

| Material | Amount (g) | % w/w | Function |
|---|---|---|---|
| Purified water | 1993.3 | 92.0 | Solvent (removed in |
| | | | process) |
| Sodium lauryl sulfate | 3.5 | 0.2 | Wetting agent |
| HPMC E5 | 169.9 | 7.8 | Binder |
| Total | 2166.7 | 100.0 | — |

Apply the sub-coat granulating solution to the desired coat level. The amount of coating is also referred to as coat level, and as defined and used herein, and for the granulation a 10% coat level is desirable, such that for 1 kg of final granulated material, 900 g is the API and 100 g is the HPMC/SLS system.

It is recognized that similar processing results may be achieved with varying conditions and equipment and those presented here are example.

TABLE 5
Processing conditions for the sub-coat/granulation step.

| Process parameter | Set Point | |
|---|---|---|
| Inlet temperature | 160 | (° F.) |
| Bed temperature | 100 | (° F.) (target) |
| Fluidizing air | 30 | (cfm) |
| Atomizing air | 30 | (psi) |

| | |
|---|---|
| Spray rate | 11 (g/min) (approximately 25% of drying capacity) |

TABLE 5A
Final theoretical composition of Lasmiditan sub-coat granulation.

| Component | % w/w |
|---|---|
| Lasmiditan hemisuccinate | 90.0 |
| Sodium lauryl sulfate | 0.2 |
| HPMC E5 | 9.8 |
| Total | 100 |

The sub-coat granulation may optionally be sieved to remove remaining fines and over-granulated material. To coat 0.3 kg of lasmiditan HPMC sub-coated/granulation with a top-coat of the reverse enteric Kollicoat® Smartseal 30 D the following general procedure may be used.

TABLE 6
Equipment description for fluid bed coating
use in the top-coat application

| Equipment | Description |
|---|---|
| Fluid bed coater | CPI Model 600 |
| Chamber | 4″ short |
| Partition | 2″ × 5″ × 0.5″ |
| Nozzle | CPI #6 generation 2 with 1 extension |
| Fluidizing plate | W6-10-1 with 325 mesh screen |

Prepare the top-coat taste masking dispersion of Kollicoat® Smartseal 30 D in purified water as shown in Table 7.

TABLE 7
Top-coat/granulating fluid composition

| | Amount | | |
|---|---|---|---|
| Material | (g) | % w/w | Function |
| Triethyl citrate | 25.9 | 1.66 | Plasticizer |
| BHT | 1.6 | 0.10 | Antioxidant |
| Purified water | 888.8 | 57.00 | Solvent (removed in process) |
| Kollicoat ® | 518.6 | 33.26 | Taste masking polymer |
| Smartseal 30D | | | |
| Talc | 124.5 | 7.98 | Detackifier |
| Total | 1559.4 | 100 | — |

It is recognized that alternate plasticizers may be used to ensure good film formation during coating. It is also recognized that antioxidants other than BHT may be used, and/or excluded altogether if appropriate for product stability.

Apply the top-coat dispersion to the desired % coat level for the coating, preferably 37% theoretical coat level. As used herein, coating level or coat level or coating can be described as a percentage on a weight-to-weight basis, of the material being coated to the weight of the coating material. Thus a 37% theoretical coat level would be represented by 1 kg of final coated API having 630 g of granulated API and 370 g of the taste masking matrix, for example, top-coat taste masking dispersion of Kollicoat® Smartseal 30 D in purified water as shown in Table 7. Useful conditions are noted in Table 8. It is recognized that similar processing results may be achieved with varying conditions and equipment with those presented here as an illustrative example. Embodiments of the present disclosure include reverse enteric coating, preferably Kollicoat® Smartseal 30 D, wherein the coat level is, for example 20-40% coat level, preferably 30-40% coat level, more preferably about 31-38% coat level, using the conditions described herein. Preferred embodiments of the invention are 32% coat level and or 37% coat level. Particularly preferred is a coat level of 37%. Coating or coated as used herein refers to the coat level and associated methods and specifications.

TABLE 8
Equipment description for fluid bed coating
use in the top-coat application

| Process parameter | Set Point | |
|---|---|---|
| Inlet temperature | 130 to 134 | (° F.) |
| Bed temperature | 84 to 86 | (° F.) (target) |
| Fluidizing air | 12 | (cfm) |
| Atomizing air | 20 | (psi) |
| Spray rate | 4.1 | (g/min) |

(approximately 40% of drying capacity)

TABLE 9
Final theoretical composition of lasmiditan hemisuccinate
taste masked at a 37% target coat level.

| Component | % w/w |
|---|---|
| Lasmiditan hemisuccinate | 56.70 |
| Kollicoat ® Smartseal 30D (on a dry | 18.72 |
| basis) | |
| Triethyl citrate | 3.12 |
| BHT (Butylated hydroxytoluene) | 0.19 |
| Talc | 14.98 |
| HPMC E5 | 6.17 |
| Sodium lauryl sulfate | 0.13 |
| Total | 100.0 |

The final coated material may optionally be further dried at temperature of 30 to 45° C. in the fluid bed coater to remove residual water and improve the quality of the coating. The final coated material may optionally be sieved to remove remaining fines and/or agglomerated material. Additional talc may be blended in with the coated API to prevent caking upon storage. Taste masking performance and subsequent release of drug in the GI tract may be modeled by measuring the API released from a representative dosage form using a USP II paddle dissolution apparatus with a pH shift method. Representative tablet dosage forms were first prepared as shown in Tables 10 and 11.

TABLE 10
Unit formula of representative ODT containing
coated (taste masked) lasmiditan.

| Ingredient | mg/tablet | % w/w | g/batch |
|---|---|---|---|
| Kollicoat ® Smartseal 30 D Coated | 196.30 | 39.26 | 9.82 |
| Lasmiditan Hemisuccinate (37% coat | | | |
| level)* | | | |
| Pharmaburst ® 500 (SPI Pharma) | 298.70 | 59.74 | 14.94 |
| Sodium Stearyl Fumarate (SPI Pharma) | 5.00 | 1.00 | 0.25 |
| Total | 500.00 | 100.00 | 25.00 |

*Equivalent to 100 mg lasmiditan
Blends were prepared and blended in a 125 mL vessel for 9 minutes at 44 rpm using a Turbula mixer. ODTs of 100 mg Lasmiditan were compressed at about 90 MPa compression stress using a Natoli single station manual tablet press and 12 mm round concave tooling.

TABLE 11
Unit formula of representative ODT containing
non-coated (non-taste masked) lasmiditan

| Ingredient | mg/tablet | % w/w | g/batch |
|---|---|---|---|
| lasmiditan hemisuccinate (uncoated)* | 115.60 | 23.12 | 5.78 |
| Pharmaburst ® 500 (SPI Pharma) | 384.40 | 76.88 | 19.22 |
| Total | 500.00 | 100.00 | 25.00 |

*Equivalent to 100 mg Lasmiditan

Blends were prepared and blended in a 125 mL vessel for 9 minutes at 44 rpm using a Turbula mixer. ODTs of 100 mg Lasmiditan were compressed at about a 35 MPa compression stress using a Natoli single station manual tablet press and 12 mm round concave tooling.

To evaluate taste masking and release properties, an ODT was placed into 900 mL of 10 mM Na phosphate/15 mM NaCl dissolution media. This media was selected as it represents the pH (about 6.5) and salinity of human saliva. While stirring at 100 rpm at 37° C. the release of lasmiditan from the dosage form was monitored every 10 seconds by measuring the UV absorption at 259 nm. After 300 seconds, 1.5 mL of 5N HCl was added to the dissolution vessel to reduce the pH to about pH 2.6 to mimic the transition to the gastric compartment.

The dissolution results as shown in Table 12 demonstrate the suppressed dissolution of lasmiditan in simulated saliva when coated with Kollicoat® Smartseal to a 37% target coat level. Similarly, the results demonstrate that upon a pH transition to about 2.6, there is rapid release of the drug from the dosage form. This is the desired release profile to ensure good taste masking and rapid release in the GI tract to ensure drug absorption.

TABLE 12
In-vitro dissolution results for representative ODTs made
using taste masked and non-taste masked drug substance.

Concentration of Lasmiditan (μg/mL)

| | | 100 mg ODT | 100 mg ODT with |
|---|---|---|---|
| | Time | with uncoated | Smartseal coated API |
| | (sec) | API | 37% CL |
| Simulated | 0 | 0.00 | 0.00 |
| saliva | 10 | 0.30 | 0.01 |
| (pH 6.5) | 20 | 24.66* | 0.10* |
| | 30 | 53.98 | 0.34 |
| | 40 | 78.41 | 0.70 |
| | 50 | 90.62 | 1.18 |
| | 60 | 95.87 | 1.56 |
| | 70 | 98.46 | 1.94 |
| | 80 | 100.04 | 2.36 |
| | 90 | 100.98 | 2.77 |
| | 100 | 101.61 | 3.23 |
| | 110 | 101.95 | 3.71 |
| | 120 | 102.19 | 4.16 |
| | 150 | 102.80 | 5.86 |
| | 180 | nm | 7.71 |
| | 210 | nm | 9.73 |
| | 240 | nm | 11.98 |
| | 300 | nm | 17.51 |
| pH 2.6 | 360 | nm | 73.88 |
| | 420 | nm | 106.28 |
| | 480 | nm | 106.79 |

*Tablet completely disintegrated in dissolution bath.
nm = not measured, CL = coat level.

### Example 2

### Manufacturing Process for Manufacture of Coated Lasmiditan

### Preparation of Coated Lasmiditan

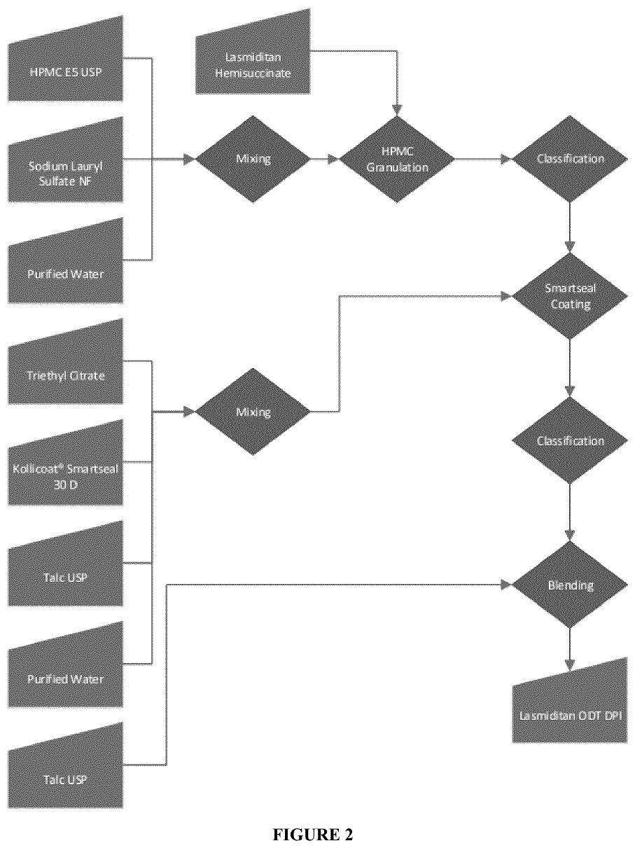

The present disclosure provides a drug product comprising an orally disintegrating tablet with dosage strengths from 25-200 mg, including 25 mg, 50 mg, 100 mg and 200 mg. A manufacturing process for manufacture of coated lasmiditan is herein provided for lasmiditan hemisuccinate which is film coated for the purpose of masking its taste prior to incorporation into orally disintegrating tablets. The lasmiditan hemisuccinate undergoes two coating steps in a Wurster style bottom spray fluidized bed coater at the 18″ scale. A process flow chart and illustrative process controls, parameters, and process ranges are described. The lasmiditan drug product intermediate manufacturing process consists of three main processes. These operations are HPMC granulation, Smartseal coating, and talc blending. The process used to manufacture the lasmiditan ODT drug product intermediate is shown in FIG. 2.

### HPMC Granulation:

The principal objective of the HPMC granulation process is to agglomerate the fine particles of the active pharmaceutical ingredient to control the particle size distribution going into the subsequent taste mask coating. The HPMC granulation process consists of the following steps outlined below.

### HPMC Solution Preparation:

Prepare HPMC solution (8% w/w solids) with an appropriate excess (if necessary) to allow for setup of liquid addition system and losses. Fill a vessel with purified water. Dissolve the HPMC in the purified water with the aid of a mixer providing a medium vortex. Once the HPMC is visually dissolved, reduce the mixer speed to provide a small vortex and continue mixing to deaerate the solution. Increase agitation speed to provide a medium vortex and add the sodium lauryl sulfate to the HPMC solution. Once all solids are visually dissolved, reduce the mixing speed to provide a low vortex during suspension deaeration. Turn the mixer off. QS the solution to its final weight with purified water. Mix solution at a low vortex for a minimum of five and a maximum of ten minutes to homogenize the solution. Turn off the mixer.

### HPMC Granulation:

Prepare 18″ Wurster-type coater by installing specified coater chamber, base plate and plate screen, nozzle, partition, plenum distribution plate, and filters. Prepare classifier by installing specified screens. Calculate amount of HPMC solution to deliver (target will result in a theoretical 10% coat level for the HPMC granulation step). Preheat the empty coater using the process parameters specified in the batch record. Fill the solution delivery line and tare the scale. Lower the coater cart and charge with lasmiditan hemisuccinate. Close the cart and adjust process parameters to the coating parameters specified in the batch record. Adjust the inlet temperature to achieve the specified target bed temperature. Once the target amount of solution has been delivered, adjust the coater parameters to the specified values for drying and dry the granulation as specified. Transfer the granulation to a drum and collect knock down fines from the coater separately. Sieve the granulation using a 249 micron screen to eliminate agglomerates and a 75 micron screen to eliminate fines.

### Kollicoat® Smartseal 30 D Coating:

The principal objective of the Kollicoat® Smartseal 30 D coating process is to apply a polymer coating to the HPMC granulation for the purpose of taste masking the material. The Kollicoat® Smartseal 30 D coating process consists of the following steps outlined below.

Kollicoat® Smartseal 30 D Coating Suspension Preparation: Prepare Kollicoat® Smartseal 30 D suspension (19.71% w/w solids) with an appropriate excess (if necessary) to allow for set-up of liquid addition system and losses. Fill a vessel with purified water. Set agitation speed to 50 RPM. Slowly add triethyl citrate to the water while agitating at this speed. Slowly add Kollicoat® Smartseal 30 D to the water/TEC mixture, passing it through a 60 mesh screen. Continue to mix at a medium vortex without introducing foam for a minimum of 90 minutes from the end of the completion of the addition of the Kollicoat® Smartseal 30 D. Increase agitation speed to provide a medium vortex and add talc to the suspension. Continue to mix using a medium vortex for a minimum of 30 minutes following the completion of the talc addition. Turn the mixer off and QS the suspension to its final weight with purified water. Mix the final suspension for a minimum of 5 minutes at a low vortex. Continue to mix suspension at a low vortex throughout the coating operation.

Kollicoat® Smartseal 30 D Coating: Prepare 18″ Wurster-type coater by installing specified coater chamber, base plate and plate screen, nozzle, partition, plenum distribution plate, and filters (For example, setup may be: Chamber is 18″ 375C, Plate is W18-10, Plate Screen is 325 mesh, Nozzle is CPI nozzle with no. 2 tip, Partition is 8.5″ x 20″ mounted 1.5″ above the plate, Plenum Distribution Plate is 1×Spoke Plate/1×Perforated Plate with 1/16″ hole diameter, and filters are 16×48″ 16 ounce PTFE). Prepare classifier by installing specified screens. Calculate amount of Kollicoat® Smartseal 30 D suspension to deliver. Preheat the empty coater using the process parameters specified in the batch record. Fill the solution delivery line and tare the scale. Lower the coater cart and charge with classified HPMC granulation. Close the cart and adjust process parameters to the coating parameters specified in the batch record. Adjust the inlet temperature to achieve the specified target bed temperature. Once the target amount of suspension has been delivered, adjust the coater parameters to the specified values for the curing step and cure the coated API as specified. Transfer the coated API to a drum and collect knock down fines from the coater separately. Sieve the coated API using a 300-micron screen to eliminate agglomerates and a 75-micron screen to eliminate fines.

Talc Blending: The principal objective of the talc blending step is to dust the coated API with a small amount of talc. This is done to mitigate extended disintegration times for tablets stressed at high temperatures. These extended disintegration times are due to agglomerates retained on the disintegration basket screen. The coated API is dusted with approximately 2% w/w talc in a diffusion blender. Use the actual weight of the coated API to calculate the required quantity of talc. Talc blending may be done in one step or in sections. Talc should be sandwiched between API additions for each section in order to minimize loss of talc on the inside surfaces of the blender. Charge approximately half of the coated API into the blender. Add the talc to the blender, and then charge the remaining coated API. Blend the mixture using the speed and time parameters specified in the batch record. Discharge the final DPI material into the specified bulk packaging containers.

Unit Formula for the lasmiditan ODT drug product intermediate:

To illustrate an embodiment of the present disclosure, a theoretical composition for the lasmiditan ODT drug product intermediate is shown in Table 13. Composition information provided in this table is theoretical based on 100% process efficiency. Composition of manufactured drug product intermediate may vary as much as ±10% during development due to scale accuracies and coating efficiencies. Lasmiditan hemisuccinate drug substance is manufactured as a single polymorphic form (anhydrous, referred to as Form A) for the coating processes described herein.

TABLE 13
Lasmiditan ODT Drug Product Intermediate
Theoretical Composition ^{A}

| | Quantity | |
|---|---|---|
| Component | (% w/w of DPI) | Function |
| Lasmiditan Hemisuccinate | 55.547 | Active Ingredient |
| Purified Water USP | —^{B} | Process Water |
| Hypromellose (HPMC) | 6.018 | Binder/API |
| | | Subcoat Polymer |
| Sodium Lauryl Sulfate | 0.154 | Wetting Agent |
| Triethyl Citrate | 2.759 | Plasticizer |
| Kollicoat ® Smartseal 30 D | 18.593^{C} | Taste Mask |
| | | Polymer Coating |
| Talc USP (1656 BC) | 14.896^{D} | Detackifier/Glidant |
| Talc USP (1656 BC) | 2.034^{E} | Detackifier/Glidant |

^{A} Composition information provided in the above table is theoretical based on 100% process efficiency. Composition of manufactured drug product intermediate may vary as much as + 10% during development due to scale accuracies and coating efficiencies.
^{B}Purified water is used in the both the HPMC granulation and the Kollicoat ® Smartseal 30 D coating operation. A majority of this water is removed during drying/curing.
^{C}Represents the solid portion of the Kollicoat ® Smartseal 30 D suspension. Kollicoat ® Smartseal 30 D is an aqueous suspension containing 30% solid components by weight.
^{D}Represents the talc present in the Kollicoat ® Smartseal 30 D coating suspension.
^{E}Represents the talc used in the final blending step of the coated composition manufacturing process.

### Batch Formula for the Lasmiditan ODT Drug Product Intermediate

The theoretical batch formula for the lasmiditan ODT drug product intermediate is shown in Table 14.

TABLE 14
Lasmiditan ODT Drug Product Intermediate
Theoretical Batch Formula

| Component | Quantity (kg) |
|---|---|
| Lasmiditan Hemisuccinate | 25.000^{A} |

HPMC Granulation Solution (8.00% w/w solids)^{B}

| | |
|---|---|
| HPMC E5 USP | 2.708 |
| Sodium Lauryl Sulfate | 0.069 |
| Purified Water^{C} | 31.944 |
| TOTAL | 34.722 |

Smartseal Coating Suspension (19.71% w/w solids)^{B,D}

| | |
|---|---|
| Triethyl Citrate | 1.242 |
| Kollicoat ® Smartseal 30 D^{E} | 27.893 |
| Talc | 6.704 |
| Purified Water^{C} | 46.930 |
| TOTAL | 82.770 |

Talc Blending

| | |
|---|---|
| Talc^{F} | 0.915 |
| TOTAL MASS OF DPI | 45.007 |

^{A}the amount of API charged into the HPMC granulation can be adjusted based on the assay value of the API. The theoretical free base content of the API is given by the ratios of the molecular weights (377.36/436.41 = 0.86469).
^{B}Represents the amount of solution/suspension delivered during coating. An excess of the solution/suspension may be prepared to account for priming of the delivery line, line losses, and in order to provide an adequate heel in the delivery tank.
^{C}Purified water is used in the both the HPMC granulation and the Kollicoat ® Smartseal 30 D coating operation. A majority of this water is removed during drying/curing.
^{D}The amount of Kollicoat ® Smartseal 30 D coating suspension is adjusted based on the yield following classification of the HPMC granulation. The amount is calculated to provide a theoretical Kollicoat ® Smartseal 30 D coat level of 37%.
^{E}Kollicoat ® Smartseal 30 D is an aqueous suspension containing 30% w/w solids.
^{F}The amount of talc used in the final blending step is adjusted based on the yield following classification of the Kollicoat ® Smartseal 30 D coated API. The amount of talc to be used in the final blending step is 20.763 g per kg of Kollicoat ® Smartseal 30 D coated API.

Preparation of Orally Disintegrating Tablets using Taste Masked Lasmiditan:

### Example 3

The unit and batch formula to prepare representative 100 mg Lasmiditan ODTs are shown in Table 15 for a theoretical batch size of 650 tablets.

TABLE 15
Unit formula and batch tablet for ODT.

| Ingredient | mg/tablet | % w/w | g/batch |
|---|---|---|---|
| Kollicoat ® Smartseal 30 D Coated | 201.17 | 40.23 | 130.76 |
| Lasmiditan Hemisuccinate | | | |
| (37% coat level)* | | | |
| Talc (extra fine) | 4.00 | 0.80 | 2.60 |
| Pharmaburst ® 500 (SPI Pharma) | 269.83 | 53.97 | 175.39 |
| Sodium Stearyl Fumarate (SPI Pharma) | 10.00 | 2.00 | 6.50 |
| Cherry berry flavoring | 5.00 | 1.00 | 3.25 |
| Aspartame | 10.00 | 2.00 | 6.50 |
| Total | 500.00 | 100.00 | 325.00 |

*Equivalent to 100 mg Lasmiditan

The coated API may be sieved through a #50 mesh to break up loose agglomerates and ensure the coated API is in discreet particulate form prior to further processing. The coated API and talc were weighed into a 500 mL vessel and blended on a Turbula for 18 minutes at 44 rpm.

The Pharmaburst® 500 is weighed into a separate 1000 mL vessel, with the cherry berry flavoring, aspartame sweetener, and sodium stearyl fumarate added on top of the Pharmaburst® in the vessel. The pre-blend of API and talc is then added on top. The 1 L vessel is then rotated on a Turbula mixer for about 10 minutes at 44 rpm.

The final blend was compressed on a FlexiTab single station press using 12 mm round dimpled tooling. The following compression profile was generated.

TABLE 16
Compression profile and physical properties
for representative ODT.

Compression stress (MPa)

| | 42 | 65 | 73 | 92 | 109 |
|---|---|---|---|---|---|
| Solid fraction (%) | 0.71 | 0.75 | 0.76 | 0.79 | 0.81 |
| Tensile strength | 0.2 | 0.4 | 0.5 | 0.8 | 1 |
| (MPa) | | | | | |
| Disintegration (sec) | 18 | 18 | 19 | 20 | 24 |
| Friability (%) | 6.4 | 1.04 | 0.43 | 0.18 | 0.1 |

The results show that with as little at 65 MPa compression stress, tablets of sufficient strength are generated to meet the target 1.0% friability target in USP<1216>. It is further recognized USP [symbol] 1216 [symbol] test may not be appropriate for ODTs; however it is a recognized and accepted characterization test. Acceptable performance in this test would be recognized as more than sufficient for an orally disintegrating tablet with respect to friability. The target disintegration time of not more than 30 seconds is met across the compression profile.

A surprising finding is that the use of talc not only does not have a negative effect on the disintegration performance of the ODT, but also serves to improve disintegration of ODTs when placed on stress stability. Talc is a hydrated magnesium silicate, its crystals are thin and lamellar forming, making it suitable as a lubricant and detackifying agent in pharmaceutical applications. Its main characteristic is that it is naturally hydrophobic and lipophilic, which would generally be thought to have a negative impact on disintegration performance, if used in a dosage form at high levels.

The following coated API-talc blends were prepared by weighing the components into a 20 mL glass scintillation vial and blending on a Turbula mixer for 40 minutes at 44 rpm.

TABLE 17
Coated API/talc pre-blend formulas.

| | Preblend | | Preblend | | Preblend | | Preblend | |
|---|---|---|---|---|---|---|---|---|
| | (0.5% talc) | | (1.0% talc) | | (2.0% talc) | | (4.0% talc) | |
| | Mass | | Mass | | Mass | | Mass | |
| | (g) | wt % | (g) | wt % | (g) | wt % | (g) | wt % |
| Coated | 3.208 | 99.504 | 3.207 | 98.951 | 3.214 | 97.958 | 3.206 | 96.017 |
| API | | | | | | | | |
| Talc | 0.016 | 0.496 | 0.034 | 1.049 | 0.067 | 2.042 | 0.133 | 3.983 |
| extra | | | | | | | | |
| Fine | | | | | | | | |
| grade | | | | | | | | |
| Total | 3.224 | 100 | 3.241 | 100 | 3.281 | 100 | 3.339 | 100 |

Pharmaburst® 500 was weighed into a 2 ounce glass jar, followed by sodium stearyl fumarate and then the coated API or coated API-talc pre-blend as added on top. This blend was rotated on a Turbula mixer for 9 minutes at 44 rpm.

TABLE 18
Unit formulas for evaluation of impact of talc on disintegration time.

| | 0% talc pre-blend | | | 0.5% talc pre-blend | | | 1% talc pre-blend | | |
|---|---|---|---|---|---|---|---|---|---|
| | per | | | per | | | per | | |
| | tablet | % | Batch | tablet | % | Batch | tablet | % | Batch |
| | (mg) | (w/w) | (mg) | (mg) | (w/w) | (mg) | (mg) | (w/w) | (mg) |
| Coated API | 200.00 | 40.00 | 3100.00 | 0.00 | 0.00 | 0.00 | 0.00 | 0.00 | 0.00 |
| Preblend | 0.00 | 0.00 | 0.00 | 201.00 | 40.20 | 3115.45 | 202.12 | 40.42 | 3132.86 |
| Pharmaburst ® | 290.00 | 58.00 | 4495.00 | 289.01 | 57.80 | 4479.66 | 287.88 | 57.58 | 4462.12 |
| 500 | | | | | | | | | |
| SSF | 10.00 | 2.00 | 155.00 | 10.00 | 2.00 | 155.00 | 10.00 | 2.00 | 155.00 |
| Total | 500.00 | 100.00 | 7750.00 | 500.01 | 100.00 | 7750.11 | 500.00 | 100.00 | 7749.99 |

TABLE 18
Unit formulas for evaluation of impact of talc on disintegration time.

| | 2% talc pre-blend | | | 4% talc pre-blend | | |
|---|---|---|---|---|---|---|
| | per | | | per | | |
| | tablet | % | Batch | tablet | % | Batch |
| | (mg) | (w/w) | (mg) | (mg) | (w/w) | (mg) |
| Coated API | 0.00 | 0.00 | 0.00 | 0.00 | 0.00 | 0.00 |
| Preblend | 204.17 | 40.83 | 3164.62 | 208.30 | 41.66 | 3228.59 |
| Pharmaburst ® 500 | 285.83 | 57.17 | 4430.37 | 281.71 | 56.34 | 4366.51 |
| SSF | 10.00 | 2.00 | 155.00 | 10.00 | 2.00 | 155.00 |
| Total | 500.00 | 100.00 | 7749.99 | 500.01 | 100.00 | 7750.10 |

Tablets were compressed at 9 kN using 12 mm round dimpled tooling using a Natoli single station press. Tablets were stressed at 70° C. open dish for the specified period of time. Tablets were removed from the oven and held at room temperature until time of analysis. Disintegration was performed per USP [symbol] 711 [symbol] in replicates of at least 3.

TABLE 19
Disintegration times (first tablet and last tablet to disintegrate)
for ODTs prepared with coated API or coated API/talc pre-blend.

| Time at | | Disintegration time (seconds) | | | | |
|---|---|---|---|---|---|---|
| 70° C. | | 0% | 0.5% | 1% | 2% | 4% |
| (hrs) | | Talc | Talc | Talc | talc | talc |
| 0 | First to | 17 | 15 | 16 | 15 | 15 |
| | disintegrate | | | | | |
| | Last to disintegrate | 17 | 15 | 16 | 15 | 15 |
| 2.25 | First to | 120 | 120 | 22 | 18 | 18 |
| | disintegrate | | | | | |
| | Last to disintegrate | 120 | 120 | 27 | 25 | 23 |
| 5.25 | First to | 120 | 120 | 34 | 23 | 25 |
| | disintegrate | | | | | |
| | Last to disintegrate | 120 | 120 | 120 | 26 | 25 |
| 21 | First to | 120 | 120 | 40 | 28 | 25 |
| | disintegrate | | | | | |
| | Last to disintegrate | 120 | 120 | 115 | 40 | 31 |

Surprisingly, despite the hydrophobic nature of talc, the disintegration performance is not compromised for unstressed tablets. When used at levels of 1% or greater, improvement in disintegration stability is obtained for tablets exposed to extreme temperature stresses.

Taste studies of the lasmiditan ODT composition as described herein or known to the skilled artisan indicate that Cherry/Berry & Aspartame flavor system has a high overall flavor quality, Bitterness & Green Stemmy attributes of flavored formulations are considerably lower than unflavored coated granules, and that chewing of a unit, in the event a patient chews against label instructions, do not change the flavor quality profile. A challenge for the compositions and tablets of the present disclosures is prevent lasmiditan hemisuccinate from going into solution while in the mouth yet ensure that it dissolves rapidly in the stomach so to achieve the required efficacy with an onset of action generally comparable to the approved tablet version. A clinically successful orally disintegrating tablet for lasmiditan aims to be palatable, bioequivalent to the approved REYVOW® tablet product forms, and consistently manufacturable.

The first hurdle to enable an ODT product form was to prepare core drug substance particles for coating wherein the particles were of the size 75 μm to 250 μm, thus being large enough to coat while also small enough to not feel gritty in the mouth on administration of an orally disintegrating tablet containing the coated particles. Drug substance batches were found to meet particle size criteria enabling the composition to use lasmiditan hemisuccinate as the core for further coating rather than resorting to more elaborate formulation approaches. Development experiments were conducted to determine if lasmiditan hemisuccinate particles could be coated by fluid bed processes, and obtain good coverage of the coating on the core, minimal or acceptable losses to the coating process. The procedures described in the present examples were determined to meet these criteria.

The compositions and orally disintegrating tablets of the present disclosure arise from the discovery of a successful barrier coat which facilitates both oral disintegration while at the same time effectively masking the highly offensive taste properties of lasmiditan. A functional coating, Kollicoat® Smartseal, was employed to mask lasmiditan API containing core particles to provide a useful degree of suppression of dissolution in the mouth. To achieve clinically tolerable taste and palatability for the target dose strengths of 50 and 100 mg, the desired product will generally result in free (solubilized) drug in the oral cavity of <1% of the administered dose. In order to achieve bioequivalence, the desired product will generally result in rapid dissolution in the gastrointestinal tract, thus providing for good absorption of lasmiditan. While numerous technologies and approaches exist for task masking, it cannot be predicted prior to clinical testing, which if any will adequately meet numerous criteria for a clinically advantageous and useful product. To achieve the desired performance characteristics, an optimal coating excipient would be insoluble at pH above 5.5 but also highly soluble at pH below 5.5. It was discovered that Kollicoat® Smartseal 30 D can be used in combination with lasmiditan for this purpose and provides superior taste masking for this API in orally disintegrating tablets. To provide the compositions capable of serving as an orally disintegrating tablet, conditions and procedures had to further be tested to determine if the lasmiditan hemisuccinate particles coated with Kollicoat® Smartseal could also be effectively tableted. Kollicoat® Smartseal coated API had an acceptable processing range, masking capacity, and made effective and useful tablets meeting required specifications.

### Example 4

### Manufacturing Process for Lasmiditan Orally Disintegrating Tablet's

The present disclosure provides embodiments of drug products comprising an orally disintegrating tablet with lasmiditan dosage strengths from 25-200 mg, including 25 mg, 50 mg, 100 mg, and 200 mg for oral administration. A manufacturing process for manufacture of lasmiditan orally disintegrating tablets is herein provided, as illustrated for lasmiditan hemisuccinate, and conceived to be useful for all forms of lasmiditan, wherein the product is film coated for the purpose of masking its taste, prior to incorporation into orally disintegrating tablets. A process flow chart and illustrative process controls, parameters, and process ranges are described.

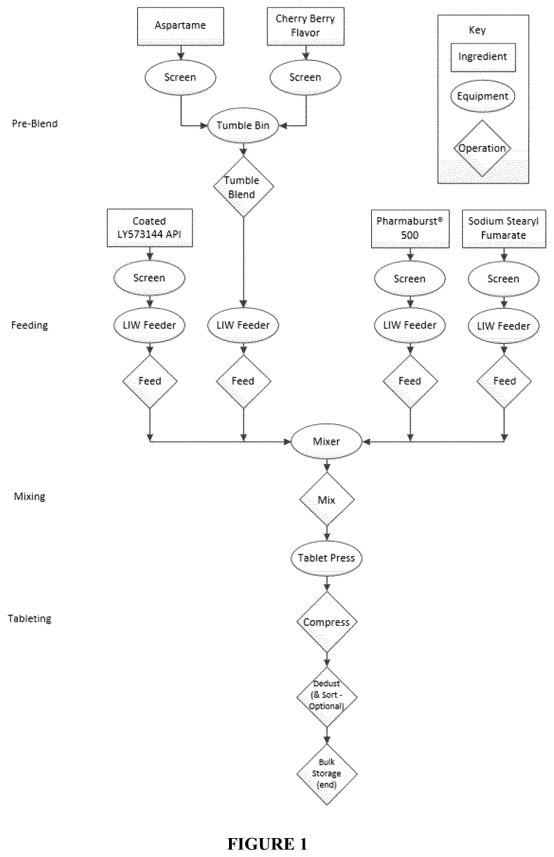

The following Table 20 provides unit formulas for Kollicoat® Smartseal 30 D Coated lasmiditan hemisuccinate Drug Product Intermediate, and 50 mg and 100 mg examples of orally disintegrating tablets. The skilled artisan may change the amounts to prepare for example 25 mg and/or 200 mg or other desired unit dosage form tablets. The lasmiditan ODT manufacturing process is shown in FIG. 1.

TABLE 20
Lasmiditan ODT Theoretical Unit Formulas

| | | | |
|---|---|---|---|
| | Quantity | | |
| | (mg/tablet) | | |
| Component | 50 mg | 100 mg | Function |

Kollicoat ® Smartseal 30 D Coated Drug Product Intermediate (DPI) ^{B,C}

| | | | |
|---|---|---|---|
| Lasmiditan Hemisuccinate | 57.8241 | 115.6482 | Active |
| Lasmiditan Free Base | 50 | 100 | |
| Purified Water ^{D} | — | — | Process Water |
| Hypromellose (HPMC) E5 | 6.2643 | 12.5286 | Binder |
| Sodium Lauryl Sulfate | 0.1606 | 0.3212 | Wetting Agent |
| Triethyl Citrate | 2.8717 | 5.7433 | Plasticizer |
| Kollicoat ® Smartseal 30 D ^{E} | 19.3550 | 38.7099 | Taste Mask |
| | | | Polymer Agent |
| Talc USP (1656 BC) (Fluid | 15.5069 | 31.0139 | Detackifier/ |
| bed coating) | | | Glidant |
| Talc USP (1656 BC) | 2.1174 | 4.2349 | Detackifier/ |
| (Extragranular blend) | | | Glidant |
| Total DPI (Coated | 104.1000 | 208.2000 | Coated Active |
| lasmiditan hemisuccinate | | | |
| API) | | | |

Oral Disintegrating Tablet ^{F}

| | | | |
|---|---|---|---|
| Coated lasmiditan | 104.1 | 208.2 | Coated Active |
| hemisuccinate API (DPI) ^{G} | | | |
| Pharmaburst ® 500 ^{G} | 131.15 | 262.3 | Disintegrant |
| Aspartame | 7.500 | 15.00 | Sweetener |
| Cherry Berry Flavor | 3.500 | 7.000 | Flavoring |
| | | | Agent |
| Sodium Stearyl Fumarate | 3.750 | 7.500 | Lubricant |
| Total Tablet Weight | 250.0 | 500.0 | |

^{A} Unit formula provided as illustrative example.
^{B} Composition and theoretical unit formula information provided in the drug product intermediate portion is theoretical based on 100% process efficiency. Composition of manufactured drug product intermediate may vary as much as ±10% during development due to scale accuracies and coating efficiencies.
^{C} Drug product intermediate is manufactured as described herein, and/or according to methods known to the skilled artisan.
^{D} Purified water is used during the drug product intermediate process and removed during the process.
^{E} Kollicoat ® Smartseal 30 D (commercially available from BASF) is an aqueous suspension containing a nominal 30 w/w % solid components and the amounts given in table are the solid portion of the suspension.
^{F} A reasonable variation of ±10% is allowed for each oral disintegrating tablet excipient unless otherwise stated.
^{G} The quantity of coated hemisuccinate API will be adjusted based on the “as-is” or standard release potency. The quantity of Pharmaburst ® 500 will be adjusted to maintain target tablet weight.

Acceptable ranges of components fed amounts per feeder as a percent of the total tablet amount are listed in Table 11. For drug substance, the range is based on maintaining a unit dose average assay value of not more than 110% and not less than 90%. For the excipients, the ranges are based on scientific judgement of ±10% reasonable variation around the target. Calculation of values are within the knowledge of the skilled artisan.

TABLE 11
Acceptable Component Fed Amount Ranges

| | Target | Minimum | Maximum |
|---|---|---|---|
| | (% of | (% of | (% of |
| Component | tablet) | tablet) | tablet) |
| Coated lasmiditan | 41.64 | 37.48 | 45.80 |
| hemisuccinate API ^{A} | | | |
| Pharmaburst ® 500 | 52.46 | 47.21 | 57.71 |
| Sweetener/Flavor Pre- | 4.40 | 3.96 | 4.84 |
| Blend ^{B} | | | |
| Sodium Stearyl | 1.50 | 1.35 | 1.65 |
| Fumarate | | | |

^{A} The quantity of coated lasmiditan hemisuccinate API will be adjusted based on the “as-is” or standard release potency. The quantity of Pharmaburst ® 500 will be adjusted to maintain target tablet weight. As such, the target (% of tablet) per feeder for the coated API and Pharmaburst ® 500 will be adjusted and a ±10% reasonable variation allowed around the potency-adjusted target.
^{B} See Table 22 for pre-blend material dispensed weight ranges.

Acceptable pre-blend component dispensed amounts as a percent of the total blend weight are listed in Table 22 and based on scientific judgement of ±10% reasonable variation on both components simultaneously. Calculation of values are within the knowledge of the skilled artisan.

TABLE 22
Acceptable Pre-Blend Dispensed Weight Ranges

| | Target | Minimum | Maximum |
|---|---|---|---|
| | (% of | (% of | (% of |
| Component | blend) | blend) | blend) |
| Aspartame | 68.2 | 63.7 | 72.4 |
| Cherry Berry Flavor | 31.8 | 27.6 | 36.3 |

A Process Flow Chart for Lasmiditan ODT Drug Product manufacture is provided in FIG. 2. The following procedures further illustrate how the ODT product can be prepared. The skilled artisan will recognize that certain variations can be employed as needed for alternative processes.

Screening and Blending of Powders (Sweetener/Flavor Pre-Blend): Aspartame and cherry berry flavor are security screened through a US standard #6 mesh sieve. Materials are layered by sequentially adding the ingredients as follows into the tumble bin: approximately half of the aspartame, all of the cherry berry flavor, the remaining aspartame. The tumble bin is placed on a tumble bin base and blended. Prior to, or while, loading material into the loss-in-weight (LIW) feeders, coated lasmiditan hemisuccinate API, Pharmaburst® 500, and sodium stearyl fumarate are security screened through a US standard #6 mesh sieve. LIW feeder material assignments and set-up configurations are listed in Table, with the preferred configuration items in bold.

TABLE 23
LIW Feeder Configurations

| | | | | Location/ | |
|---|---|---|---|---|---|
| Feeder | | | Screw | Mixer | Outlet |
| # | Material | Size | Type | Inlet ^{A} | Screen |
| 1, 2 or | Coated | T20 or | Fine or | Upper/1 | None |
| 4 | lasmiditan API | T35 | Coarse | | |
| | | | Stainless | | |
| 3 | Pharmaburst ® | T35 | Fine or | Upper/1 | None |
| | 500 | | Coarse | | |
| | | | Stainless ^{B} | | |
| 5 | Sodium Stearyl | T20 | Fine or | Middle/3 | None |
| | Fumarate | | Coarse | | |
| | | | Stainless | | |
| 6 | Sweetener/Flavor | T20 | Fine or | Middle/2 | None |
| | Pre-Blend | | Coarse | | |
| | | | Stainless | | |

^{A} A continuous manufacturing suite is setup for mixing by methods known to the skilled artisan. Table 24 below illustrates equipment and setup parameters.

TABLE 24
Equipment List/Setup

| | | | |
|---|---|---|---|
| | | Equipment | |
| Equipment | Manufacturer | Parameter | Recommended Specification ^{A} |

Sweetener/Flavor Pre-Blend

| | | | |
|---|---|---|---|
| Screen (security) ^{B} | Tyler or equivalent | Screen Mesh | US Std #6 (3350 micron) |
| Diffusion Bin | L. B. Bohle, Germany | Bin Volume | 1.4 ft³ (nominal 1 ft³) |
| | (or equivalent) | Dimensions | Rectangle, Conical bottom |
| | | Discharge Angle | 90° (vertical) |
| | | Axis of rotation | Horizontal |

Feeding

| | | | |
|---|---|---|---|
| Screen (security) ^{B} | Tyler or equivalent | Screen Mesh | US Std #6 (3350 micron) |
| Feeder for | Coperion K-Tron, | Loss in Weight | T35 LIW Feeder |
| Pharmaburst ® 500 | Germany | Feeder/Scale | |
| Feeders for | Coperion K-Tron, | Loss in Weight | T20 LIW Feeder |
| sweetener/flavor pre- | Germany | Feeder/Scale | |
| blend and sodium | | | |
| stearyl fumarate | | | |
| Feeder coated | Coperion K-Tron, | Loss in Weight | T20 or T35 LIW Feeder |
| LY573144 | Germany | Feeder/Scale | |
| hemisuccinate API | | | |

Mixing

| | | | |
|---|---|---|---|
| Mixer | Gericke, Switzerland | Model | GCM350 or GCM450 |
| | | Weir | 180°, fully open |
| | | Volume | 8 L |
| | | Paddle | Paddle 1 and 12 at 45° forward. |
| | | Configuration | Paddles 2-11 at 22.5°, odd |
| | | | numbered paddles facing |
| | | | outlet/forward, even numbered |
| | | | paddles facing inlet/backward. |

Surge Hopper

| | | | |
|---|---|---|---|
| Hopper | Lilly Design, USA | Materials of | FDA compliant materials |
| | | Construction | |
| Level Sensor | Fluidwell, The | Model | Triflex LNI 200 |
| | Netherlands | Measurement | 20 capacitance sensors |

Tableting

| | | | |
|---|---|---|---|
| Tablet Press - Power | Korsch, Germany | Model | Korsch XL200 |
| Assisted | | Number of Stations | Fully tooled |
| | | Used | |
| | | Turret Pitch | 285 mm |
| | | Diameter | |
| | | Feeder Paddle | 3 paddle, square profile |
| | | Design | |
| | | Fill cam size | 10 mm |
| | | Tooling Design, | 50 mg: Ø 9.50 mm (0.3740 |
| | | round dimple flat- | inches) |
| | | faced, beveled edge | 100 mg: Ø 12.00 mm (0.4724 |
| | | (FFBE) embossed | inches) |
| | | Punch Head Design | TSM-B Domed Head |

^{A} Recommended specification based on typical equipment capability/specifications from manufacturers and scientific judgement.
^{B} Materials are screened prior to loading in feeders.

TABLE 24
Equipment List/Setup (cont.)

| | | | |
|---|---|---|---|
| | | Equipment | |
| Equipment | Manufacturer | Parameter | Recommended Specification ^{A} |

Powder Near-infrared

| | | | |
|---|---|---|---|
| Spectrometer | Prozess, USA | Model | Prozess 611 NIRS |
| | | Spectrometer Type | Diode-Array |
| | | Nominal | Approx. 1100-2100 nm |
| | | Wavelength Range | |
| | | Wavelength | Approx. 5 nm |
| | | Spacing | |
| | | Scan Time | Approx. 1.2 sec |
| | | System Software | NovaPAC |
| | | Probe Type | Flat head, 6 around 1 fiber optic |

Tablet Tester

| | | | |
|---|---|---|---|
| Tandem | Bruker, Germany | Model | Tandem IIIA |

^{A} Recommended specification based on typical equipment capability/specifications from manufacturers.

TABLE 22
Process Parameters and Recommended Ranges

| | | |
|---|---|---|
| | | Recommended |
| Parameter | Target | Range ^{A} |

Pre-Blend

| Fill Level | N/A | | 15-75% | |
|---|---|---|---|---|
| | | | 1.8-8.5 Kg | |
| Speed | 12 | rpm | 10-15 | rpm |
| Time | 8.3 | min | 6.7-10 | min |

Continuous Processing

| Total Flow | 32.4 | kg/h | 25.0-40.0 kg/h | |
|---|---|---|---|---|
| | | | (based on FF NIR | |
| | | | calibration) | |
| Mixer Impeller | 150 | rpm | 125-175 | rpm |
| Speed | | | | |
| Compression Dwell | 50 mg: 7.1 ms | | 50 mg: 6.4-7.9 ms | |
| Time | 100 mg: 14.2 ms | | 100 mg: 11.6-18.2 ms | |
| Turret Speed | 50 mg: 90 rpm | | 50 mg: 80-100 rpm | |
| | 100 mg: 45 rpm | | 100 mg: 35-55 rpm | |
| | | | (based on FF NIR | |
| | | | calibration) | |
| Tablet Press Feeder | ~½ of turret speed, | | 15-50 | rpm |
| Speed | adjust to minimize main | | (based on FF NIR | |
| | compression force RSD | | calibration) | |
| | (Srel) at start-up | | | |
| Fill Depth | Adjust to target tablet | | n/a | |
| | weight | | | |
| Pre-Compression | Adjust to achieve ~10% | | n/a | |
| Force | of main compression | | | |
| | force | | | |
| Pre-Compression | Adjust to desired force | | n/a | |
| Punch Tip-to-Tip | | | | |
| Height (“Edge Pre”) | | | | |
| Main Compression | Adjust to target | | n/a | |
| Punch Tip-to-Tip | thickness/solid fraction | | | |
| Height (“Edge Main”) | | | | |
| Pre-Compression | 2.5 | mm | 2.0-3.0 | mm |
| Punch Penetration | | | | |
| Main Compression | 2.5 | mm | 2.0-3.0 | mm |

Punch Penetration

^{A} Recommended range based on typical equipment capability, development experience.

Mixing: A mixer is employed wherein the mixer shaft has paddles in an alternating 22.5° configuration (odd paddles facing outlet, even paddles facing inlets) except for paddles #1 and #12 that both face the outlet at a 45° (see Table 24). The mixer is equipped with an integrated adjustable weir assembly in the outlet piece, which is used to adjust the amount of material holdup in the mixer. The weir is kept in the full open position during the product collection (runtime) phase of the process, but may be adjusted for initial process setup to assure uniformity while adjusting parameters for tablet weight and thickness. Should the weir need to be closed, the impeller speed is reduced to no more than 100 rpm such that the centripetal force is less than the inertial force of the powder inside the mixer (a Froude number less than 1).

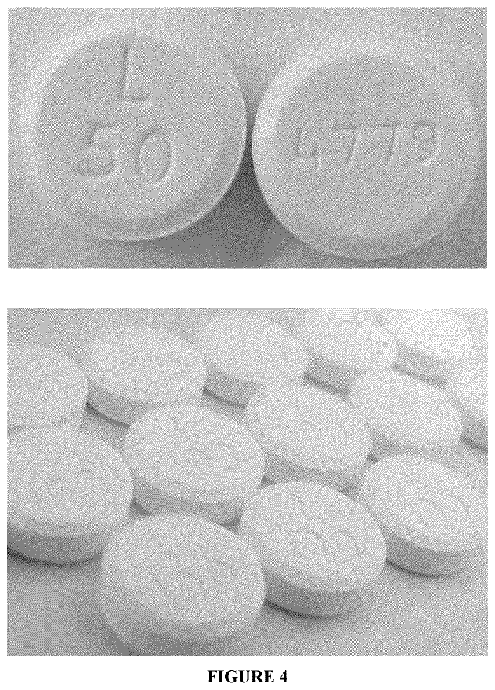

Tableting: The final blend is compressed into round dimple flat-faced, beveled edge (FFBE) tablets of dimensions given in Table using tooling HOB numbers listed. A rotary compression machine (e.g., Korsch XL200) is used to create the tablets. Tablet press production rate which determines the target turret speed is a DCS recipe parameter. All other tablet press process parameters for each tablet strength are defined by tablet press recipes. Illustrative orally disintegrating tablets (50 mg and 100 mg) are shown in FIG. 4.

TABLE 26
Tablet Dimensions and Tooling HOB Numbers

| | Dimensions (Round Ø) | | |
|---|---|---|---|
| Strength | U.S. English | Metric | HOB# |
| (mg) | (in.) | (mm) | Upper/Lower |
| 50 | 0.3740 | 9.50 | 187822/187823 |
| 100 | 0.4724 | 12.00 | 187825/187826 |

The turret speed may be adjusted to control the mass flow out of the press to match the mass flow into the press surge hopper from the mixer. This adjustment may be manual or via automation with the surge hopper level sensor to maintain to suitable column of powder throughout the steady product collection phase.

The compression parameters are configured during setup to achieve the target tablet physical attributes (listed in Table 26 and Table). The tablet press dosing is adjusted to achieve target tablet weight. The tablet press feed frame (feeder) paddle speed is adjusted to minimize main compression force RSD (“Srel”) which is an indicator of tablet weight variation. Pre-compression and main compression tooling tip-to-tip (edge) distances will be adjusted to achieve the desired tablet compact strength and/or thickness. The tablet press recipe parameters are considered initial conditions to start the process and the parameters can be adjusted as needed to obtain the desired tablet properties (such as values for dosing, edge thickness, compression force, etc.).

Tablet weight, thickness, breaking force, disintegration, and friability are evaluated at the start-up. Tablet weight and thickness, as well as the corresponding calculated solid fraction, will be routinely evaluated throughout the compression run. All tablets are passed through a tablet de-duster and metal checker. Tablets may be sorted, as needed.

### Tablet Physical Attributes

Tablets are assessed by methods known to the skilled artisan and or described herein.

### Average Tablet Weight:

Average tablet weight is measured by weighing individual tablets on a balance and calculating the average value.

### Average Breaking Force (Hardness):

Tablet breaking force is measured under loading across the diameter of the round tablets using a hardness tester. The maximum compressive load (breaking force) achieved at tablet failure is recorded for individual tablets and the average calculated. Refer to USP Guidance [symbol] 1217 [symbol] for more information.

### Average Tablet Thickness:

The largest distance between the tablet faces is measured with a micrometer, recorded for individual tablets, and the average is calculated.

### Average Solid Fraction:

Average solid fraction is calculated using Equation 1 and 2:

$$\\ \begin{matrix}Solid Fraction = \frac{Average Tablet Weight}{Tablet Volume \times True Density} & Equation 1\end{matrix}$$

Equation 1 can be further described for a given set of tablet tooling by Equation 2.

$$\\ \begin{matrix}Solid Fraction = \frac{Average Tablet Weight}{( 2 \times Cup Volume + [ \begin{matrix}Die Hole Area \times \\ ( \begin{matrix}Average Thickness - \\ 2 \times Cup Depth\end{matrix} )\end{matrix} ] ) \times True Density} & Equation 2\end{matrix}$$

Alternatively, average solid fraction can be the average of the individually calculated solid fractions for a given set of tablets, using the weight and thickness values for each tablet.

### Friability:

The total weight of de-dusted tablets is measured before and after rotating them at 25 rpm in a friability tester's drum for 100 revolutions. To ensure the accuracy of the tablet weights, tablets should be exposed to atmospheric room conditions prior to testing to allow for equilibration with ambient conditions. The resulting calculated percentage weight difference is the tablet friability. Refer to USP Guidance [symbol] 1216 [symbol] for more information.

### Disintegration:

Tablets are placed on separate screens while being lowered and raised in 37±2 ° C.water bath until all tablet pieces fall through the screen. Refer to USP Guidance [symbol] 701 [symbol] .

Tablet physical attributes are to be evaluated at batch start-up and periodically during the batch to control tablet weight and monitor thickness, solid fraction, and/or tablet strength.

TABLE 26
50 mg Tablet Strength

| | | |
|---|---|---|
| Tablet Physical | Target | Recommended |
| Attributes ^{A} | Value | Range |

| | | | | |
|---|---|---|---|---|
| Average Tablet Weight* | 250 | mg | 237.5-262.5 | mg |
| Average Thickness | 3.21 | mm | 3.14-3.28 | mm |

| | | |
|---|---|---|
| Average Solid Fraction* | 0.82 | 0.80-0.84 |

| | | | |
|---|---|---|---|
| Average Tablet Breaking | 4.5 | kp | NLT 3.3 kp |
| Force | | | |

| | | |
|---|---|---|
| Tablet Friability | n/a | NMT 0.5% |
| Disintegration (last tablet) | n/a | NMT 30 s |

^{A} Tablet physical attributes used as in-process controls are marked with an asterisk (*).

TABLE 27
100 mg Tablet Strength

| | | |
|---|---|---|
| Tablet Physical | Target | Recommended |
| Attributes ^{A} | Value | Range |

| | | | | |
|---|---|---|---|---|
| Average Tablet Weight* | 500 | mg | 475-525 | mg |
| Average Thickness | 4.01 | mm | 3.92-4.10 | mm |

| | | |
|---|---|---|
| Average Solid Fraction* | 0.82 | 0.80-0.84 |

| | | | |
|---|---|---|---|
| Average Tablet Breaking | 6.9 | kp | NLT 5.1 kp |
| Force | | | |

| | | |
|---|---|---|
| Tablet Friability | n/a | NMT 0.5% |
| Disintegration (last tablet) | n/a | NMT 30 s |

^{A} Tablet physical attributes used as in-process controls are marked with an asterisk (*).
Storage Conditions: USP controlled room temperature.

Taste masking can be evaluated by Taste Profiling Procedures using a Flavor Profile Method. Sensory panelists evaluate the samples using the Flavor Profile Method of descriptive sensory analysis (Keane, P. The Flavor Profile Method. In C. Hootman (Ed.), Manual on Descriptive Analysis Testing for Sensory Evaluation ASTM Manual Series: MNL 13. Baltimore, Md. (1992)). For illustration, a Taste Profiling Procedure Tablet Evaluation Protocol is as follows: 1. The panelists cleanse their palates with spring water and unsalted crackers. 2. One lasmiditan tablet is dispensed to each panelist. 3. Starting at the same time, panelists place the tablet in the oral cavity and gently roll after being placed on top of tongue (for ODTs) or chewed (for chewable tablets) until the point at which the panelist normally would have swallowed. The material left in the mouth was then expectorated and the disintegration or chew time was recorded. 4. The panelists then independently evaluate and record the initial and aftertaste characteristics at periodic intervals up to 30 minutes as flavor persisted. 5. The panelists recite their individual results and a preliminary Flavor Profile is generated for the sample. 6. Steps 1 through 4 are repeated for a second sample using the preliminary Flavor Profile from Step 5 as a guide, with the panelists making any necessary modifications. 7. The panelists recite their individual results and a final Flavor Profile is developed for the sample.

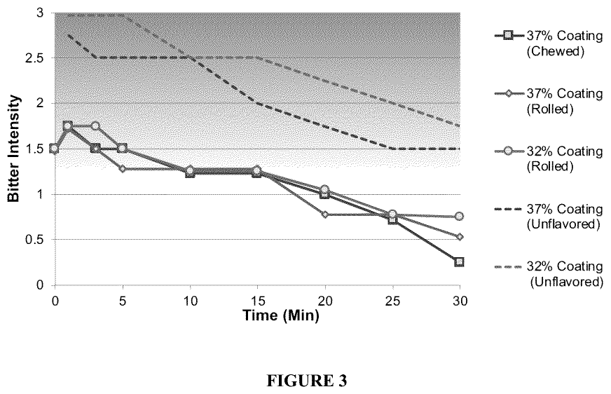

It was discovered that pairing the Kollicoat® Smartseal 30 D -coated API with a properly selected flavor & sweetener provided a palatable ODT (less than about 1.5 on bitter intensity scale). Illustrative results for resulting Flavor Profiles for lasmiditan ODT embodiments are summarized below. Flavored formulations were significantly lower in bitterness than their unflavored granules as shown in FIG. 3, Taste Profiling of Lasmiditan ODT embodiments using a Flavor Profile Method (FIG. 3 dashed lines are unflavored, solid lines are flavored). Two coating levels were compared, and 32% coat level was only very slightly more bitter than the 37% coated lasmiditan. Rolling and chewing produced resulted in equivalent bitterness profiles.

The sweetened/flavored lasmiditan ODT formulations of the present disclosure are reasonably high in overall flavor quality. The target balance and fullness for oral drug products is about 1.5 or lower, and when rolled the 37% coated flavor system achieved this target. Chewed tablets were only of marginally lower. The bitterness of lasmiditan flavored ODT formulations is considerably lower than unflavored coated lasmiditan granules. Based on flavor quality, this sweetened/favored lasmiditan formulation is suitable for an ODT product form. Patient choice to chew would not significantly change or worsen the flavor profile of the tablets (i.e., the granule coating remains mostly intact).

In addition to the taste masking provided by the coating system, a “flavor system” (sweetener and identifying aromatics) was added to the highest coat level (37%) powder blend to further improve palatability of the lasmiditan ODT. This effort resulted in preferred excipients: High Intensity (artificial) Sweetener—Aspartame, and Cherry Berry flavoring were found to offset residual bitterness and surprisingly provides a palatable lasmiditan ODT product form. Other negative sensory attributes such as tongue sting, throat burn, and mouth numbing were practically eliminated. Further, in-vitro data and modeling indicated the lasmiditan ODT formulation of the present disclosure is expected to be bioequivalent to the approved immediate release tablet, and this is being tested in clinical study LAIA (Bioequivalence of Lasmiditan Oral Disintegrating Tablet Compared to Current Immediate-Release Tablet Formulation to Support Treatment of Migraine).

### Example 5

### Comparative Example

Surprisingly the performance of an alternative but similar reverse enteric coating, Eudragit® E100, was found to be inferior to that of the Kollicoat® Smartseal, both in terms of taste masking performance and disintegration performance when processed into an ODT. EUDRAGIT® E 100 is a cationic copolymer based on dimethylaminoethyl methacrylate, butyl methacrylate, and methyl methacrylate manufactured by Evonik Health care. It is supplied as the polymer solid substance (E 100), a solution in alcohol (E 12.5) and as ready to use dry mix power. Eudragit® E is marketed as a reverse enteric polymer for taste masking applications and would be expected to perform similarly to Kollicoat® Smartseal, also a reverse enteric polymer of similar chemical class.

The following procedure can be applied to coat 0.3 kg of lasmiditan hemisuccinate of a particle size of d10=55.0 μm, d50=117.9 μm, and d90=220.9 μm or similar with Eudragit® EPO. Charge the fluid bed coater as described in Table 28 with drug substance. There are many vendors who supply fluid bed coaters capable of Wurster coating and the equipment set-up may differ between vendors, particularly with respect to nozzle type and fluidization parameters. The example cited is for one particular style of fluid bed coater, but the skilled artisan will understand that other fluid bed coaters may be used to achieve similar results.

TABLE 28
Equipment description for fluid bed coating.

| Equipment | Description |
|---|---|
| Fluid bed coater | CPI Model 600 |
| Chamber | 4″ short |
| Partition | 2″ × 5″ × 0.5″ |
| Nozzle | CPI #6 generation 2 with 1 extension |
| Fluidizing plate | W6-10-1 with 325 mesh screen |

Prepare the sub-coat/granulating solution of HPMC E5 and SLS solution in purified water as shown in Table 29.

TABLE 29
Sub-coat/granulating fluid composition.

| Material | Amount (g) | % w/w | Function |
|---|---|---|---|
| Purified water | 948.8 | 91.99 | Solvent (removed |
| | | | in process) |
| Sodium lauryl sulfate | 1.7 | 0.16 | Wetting agent |
| HPMC E5 | 80.9 | 7.84 | Binder |
| Total | 1031.4 | 100.00 | — |

Apply the sub-coat granulating solution to the desired weight gain of 5 to 15 wt % gain, preferably 10% weight gain, using the target conditions shown in Table 30. It is recognized by the skilled artisan that similar processing results may be achieved with varying conditions and equipment and those presented here are example.

TABLE 30
Processing conditions for the sub-coat/granulation step.

| Process parameter | Set Point |
|---|---|
| Inlet temperature | 151 to 183 (° F.) |
| Bed temperature | 97 to 104 (° F.) (target) |
| Fluidizing air | 11.7 to 12.3 (cfm) |
| Atomizing air | 25 to 30 (psi) |
| Spray rate | 3.5 (g/min) (approximately 20% of drying capacity) |

TABLE 31
Final theoretical composition of lasmiditan sub-coat granulation.

| Component | % w/w |
|---|---|
| Lasmiditan hemisuccinate | 90.0 |
| Sodium lauryl sulfate | 0.2 |
| HPMC E5 | 9.8 |
| Total | 100 |

The sub-coat granulation may optionally be sieved to remove remaining fines and over-granulated material. To coat 0.253 kg of Lasmiditan HPMC sub-coated/granulation with a top coat of the reverse enteric Eudragit® E PO. Prepare the top-coat taste masking dispersion of Eudragit E PO in purified water as shown in Table 32.

TABLE 32
Eudragit ® E PO top-coat fluid composition

| | Amount | | |
|---|---|---|---|
| Material | (g) | % w/w | Function |
| Stearic acid | 24.8 | 1.29 | Plasticizer |
| (Kolliwax ® S Fine) | | | |
| Sodium Lauryl sulfate | 16.5 | 0.86 | Wetting agent |
| Purified water | 1630.9 | 85.00 | Solvent (removed |
| | | | in process) |
| Eudragit ® EPO | 164.4 | 8.57 | Taste masking polymer |
| Talc | 82.1 | 4.28 | Detackifier |
| Total | 1918.7 | 100.00 | — |

Apply the top-coat dispersion to the desired weight gain of 44 wt % theoretical, (relative to the charge weight of the granulated substrate) using the conditions described in Table 33. It is recognized by the skilled artisan that similar processing results may be achieved with varying conditions and equipment and those presented here are example.

TABLE 33
Process parameters for fluid bed coating use in the
Eudragit ® E PO top coat application

| Process parameter | Set Point |
|---|---|
| Inlet temperature | 109 to 119 (° F.) |
| Bed temperature | 77 (° F.) (target) |
| Fluidizing air | 12 (cfm) |
| Atomizing air | 20 (psi) |
| Spray rate | 4.1 (g/min) (approximately 40% of drying capacity) |

TABLE 34
Final theoretical composition of Lasmiditan hemisuccinate taste masked
at a 44% target coat level with Eudragit ® E PO.

| Component | % w/w |
|---|---|
| Lasmiditan hemisuccinate | 50.40 |
| Eudragit ® EPO | 25.139 |
| Sodium lauryl sulfate (top coat) | 2.523 |
| Stearic acid (Kolliwax S fine) | 3.784 |
| Talc | 12.555 |
| HPMC E5 | 5.490 |
| Sodium Lauryl sulfate (sub-coat) | 0.112 |
| Total | 100.0 |

The final coated material may optionally be sieved to remove remaining fines and/or granulated material.
Preparation of the ODT using Eudragit® E PO Coated Drug Substance:

The unit and batch formula to prepare representative 100 mg Lasmiditan ODTs is shown in Table 35 for a theoretical batch size of 300 tablets.

TABLE 35
Unit formula and batch tablet for ODT.

| Ingredient | mg/tablet | % w/w | g/batch |
|---|---|---|---|
| Eudragit ® EPO Coated lasmiditan | 229.9 | 45.98 | 68.97 |
| hemisuccinate (44% coat level)* | | | |
| Pharmaburst ® 500 (SPI Pharma) | 267.6 | 53.52 | 80.28 |
| Sodium Stearyl Fumarate (SPI Pharma) | 2.5 | 0.5 | 0.75 |
| Total | 500.00 | 100.00 | 150 |

*Equivalent to 100 mg Lasmiditan

The coated API may be sieved through a #50 mesh to break up loose agglomerates and ensure the coated API is in discreet particulate form. The Pharmaburst® 500 is weighed into a 500 mL vessel, then sodium stearyl fumarate the n the coated API. The vessel is then rotated on a Turbula mixer for about 7 minutes at 44 rpm. The final blend was compressed on a FlexiTab single station press using 12 mm round convex tooling. The following compression profile was generated.

TABLE 36
Compression profile for ODT.

Compression stress (MPa)

| | 33 | 60 | 90 | 118 |
|---|---|---|---|---|
| Solid fraction (%) | 0.72 | 0.79 | 0.84 | 0.86 |
| Tensile strength (MPa) | 0.3 | 0.7 | 1.2 | 1.6 |
| Disintegration (sec) | 18 | 44 | 118 | 180 |
| Friability (%) | 2.21 | 0.15 | 0.02 | 0.00 |

The results show that with a 60 MPa compression stress, tablets of sufficient strength are generated to meet the target 1.0% friability; however, the disintegration time of 44 seconds at this compression stress exceeds the acceptable limit of 30 seconds. To reduce the disintegrations time to a more acceptable 18 seconds, a compression stress of 33 MPa is required, but this produces soft tablets as reflected in the high friability value of 2.21% presenting risk for manufacturing and downstream handling. Thus, there is a narrow and impractical compression operating window to manufacture tablets of adequate strength with low disintegration time.

To evaluate taste masking and release properties of the Eudragit® E PO coated API, the same dissolution procedure as described previously herein is used. Table 37 shows the pH shift dissolution profiles of ODTs prepared using the uncoated API, Kollicoat® Smartseal coated API, and Eudragit® E PO coated API. Dissolution of API from a tablet generally follows the sequence that the tablet must first disintegrate prior to API dissolution. In this case, tablets made with Eudragit® E PO are slow to disintegrate relative to the other two tablets. As such, the early dissolution time points for the Eudragit® E PO tablets show artificially low drug release, as the tablet did not fully disintegrate until around 4 minutes, compared to the preferred 20 seconds for the other tablets. In looking at the time points from about 120 seconds and beyond, when at least 50% of the Eudragit® E PO tablet has disintegrated, it is clear that the rate of release from the Eudragit® E PO coated API particles is significantly greater than that of the Kollicoat® Smartseal coated API particles. The data show that Eudragit® E PO is less effective at suppressing release of Lasmiditan hemisuccinate after complete tablet disintegration. This is even more evident at the 300 second time point, just prior to the pH shift, where the % release is 17.51% for the Kollicoat® Smartseal coated API versus 53.26% for the Eudragit® E PO coated Lasmiditan hemisuccinate.

TABLE 37
Compression profile for ODT.

| | | Concentration of lasmiditan (μg/mL) | | |
|---|---|---|---|---|
| | | | 100 mg ODT with | 100 mg ODT with |
| | Time | 100 mg ODT with | Smartseal coated | Eudragit ® EPO coated |
| | (sec) | uncoated API | API 37% CL | API 44% CL |
| | 0 | 0.00 | 0.00 | 0.00 |
| Simulated | 10 | 0.30 | 0.01 | 0.02 |
| saliva | 20 | 24.66* | 0.10* | 0.05 |
| (pH 6.5) | 30 | 53.98 | 0.34 | 0.17 |
| | 40 | 78.41 | 0.70 | 0.32 |
| | 50 | 90.62 | 1.18 | 0.53 |
| | 60 | 95.87 | 1.56 | 0.90 |
| | 70 | 98.46 | 1.94 | 1.32 |
| | 80 | 100.04 | 2.36 | 1.85 |
| | 90 | 100.98 | 2.77 | 2.50 |
| | 100 | 101.61 | 3.23 | 3.26 |
| | 110 | 101.95 | 3.71 | 4.35 |
| | 120 | 102.19 | 4.16 | 5.39** |
| | 150 | 102.80 | 5.86 | 9.54 |
| | 180 | nm | 7.71 | 15.76 |
| | 210 | nm | 9.73 | 24.15 |
| | 240 | nm | 11.98 | 34.19* |
| | 300 | nm | 17.51 | 53.26 |
| pH 2.6 | 360 | nm | 73.88 | 95.51 |
| | 420 | nm | 106.28 | 110.16 |
| | 480 | nm | 106.79 | 110.98 |

*Tablet 100% disintegrated in dissolution bath
**Tablet about 50% disintegrated at this time
nm = not measured

1. A pharmaceutical composition comprising lasmiditan, or a pharmaceutically acceptable salt thereof, and a reverse enteric coating.

2. The composition of claim 1 wherein the lasmiditan, or a pharmaceutically acceptable salt thereof, is lasmiditan hemisuccinate.

3. The composition of claim 1 or 2 wherein the lasmiditan comprises granulated particles having a size range of about 50 to about 275 microns.

4. The composition of claim 3 wherein the reverse enteric coating is Kollicoat® Smartseal 30 D which comprises methyl methacrylate-di(ethyl)aminoethyl methacrylate copolymer.

5. The composition of claim 4, wherein the composition further comprises about 20-40% coat level relative to the weight of granulated lasmiditan particles upon coating with Kollicoat® Smartseal 30 D.

6. The composition of claim 5, wherein the composition further comprises about 37% coat level relative to the weight of granulated lasmiditan particles upon coating with Kollicoat® Smartseal 30 D.

7. The composition of claim 6 wherein the lasmiditan to be coated with Kollicoat® Smartseal 30 D further comprises talc.

8. The composition of claim 7 wherein the lasmiditan to be coated with Kollicoat® Smartseal 30 D further comprises talc, and the final coated particles have a size range between about 75 and about 300 microns.

9. The composition of claim 8 further comprising Talc, Pharmaburst® 500, and Sodium Stearyl Fumarate.

10. The composition of claim 9 further comprising a sweetener and a flavoring agent.

11. The composition of claim 10 wherein the sweetener is Aspartame and the flavoring agent is Cherry berry.

12. The composition of claim 11 wherein the composition comprises:
about 37% to 46% w/w of Kollicoat® Smartseal 30 D Coated Lasmiditan Hemisuccinate,
about 47% to 58% w/w of Pharmaburst® 500,
about 3.9% to 4.9% w/w of aspartame/Cherry berry flavoring blend (Aspartame about 68% to Cherry Berry Flavor about 32% w/w); and
about 1.3% to 1.7% w/w of Sodium Stearyl Fumarate.

13. The composition of claim 11 wherein the composition comprises:
(i) about 40.2% w/w of Kollicoat® Smartseal 30 D Coated Lasmiditan Hemisuccinate (about 37% coat level),
(ii) about 0.80% w/w of Talc,
(iii) about 54.0% w/w of Pharmaburst® 500,
(iv) about 2.0% w/w of Sodium Stearyl Fumarate,
(v) about 1.0% w/w of Cherry berry flavoring, and
(vi) about 2.0% w/w of Aspartame.

14. The composition of any of claims 1 to 3, or 5 to 13, wherein the composition further comprises a dosage of lasmiditan from about 25 mg to about 200 mg.

15. The composition of claim 14 wherein the composition further comprises a dosage of lasmiditan from about 25 mg to about 100 mg.

16. The composition of claim 15 wherein the composition further comprises a dosage of lasmiditan of about 25 mg.

17. The composition of claim 15 wherein the composition further comprises a dosage of lasmiditan of about 50 mg.

18. The composition of claim 15 wherein the composition further comprises a dosage of lasmiditan of about 75 mg.

19. The composition of claim 15 wherein the composition further comprises a dosage of lasmiditan of about 100 mg.

20. The composition of claim 14 wherein the composition further comprises a dosage of lasmiditan of about 150 mg.

21. The composition of claims 1-20 wherein the composition further comprises an orally disintegrating tablet.

22. A method of treating migraine in a patient comprising administering to a patient in need of such treatment an effective amount of a composition according to any one of claims 14-20.

23. (canceled)

24. (canceled)

25. A compressed orally disintegrating tablet comprising a disintegrant and a plurality of units comprising:
i) a plurality of particles comprising a therapeutically effective amount of lasmiditan or a pharmaceutically acceptable salt thereof;
ii) a reverse enteric coating over the particles comprising a reverse enteric polymer in an amount of 20% to 40% coat level;
wherein the disintegrant and the plurality of units are compressed to an orally disintegrating tablet having a friability of 1% or less when 6 kN to 50 kN of a compression force is applied during manufacturing of the tablet.

26. A process of manufacturing the orally disintegrating tablet of claim 21 comprising:
a) generating a plurality of particles comprising a therapeutically effective amount of lasmiditan, or a pharmaceutically acceptable salt thereof;
b) applying a coating comprising a reverse enteric polymer to the particles of step (a) thereby obtaining a plurality of units;
c) mixing the plurality of units of step (b) with at least one tablet excipient comprising a disintegrant thereby obtaining a blend;
d) mixing the blend of step (c) with a flavor and a sweetener to make a taste masked blend;
e) mixing the taste masked blend with a dry lubricant; and
f) compressing the blend of step (e) thereby obtaining the compressed orally disintegrating tablet.
